(19) United States
(12) Patent Application Publication
(10) Pub. No.: US 2012/0046284 A1
(43) Pub. Date: Feb. 23, 2012

(54) Salts and hydrates of 4-[(3-chloro-4-fluoro-phenyl)amino]-6-(cis-4--cyclohexan-1-yloxy)-7-methoxy-quinazoline, their use as a medicament and the preparation thereof

(75) Inventors: Marc EGEN, Ingelheim am Rhein (DE); Werner RALL, Mittelbiberach (DE); Marco SANTAGOSTINO, Mittelbiberach (DE); Juergen SCHNAUBELT, Oberhoefen/Warthausen (DE); Peter SIEGER, Mittelbiberach (DE); Rainer SOYKA, Biberach (DE)

(73) Assignee: BOEHRINGER INGELHEIM INTERNATIONAL GMBH, Ingelheim am Rhein (DE)

(21) Appl. No.: 13/026,817

(22) Filed: Feb. 14, 2011

(30) Foreign Application Priority Data
Feb. 15, 2010 (EP) 10153572.2

(51) Int. Cl. A61K 31/5377; A61P 11/00; A61P 29/00; A61P 37/08; C07D 413/12; C07D 239/94

(52) U.S. Cl. 514/234.5; 544/119; 544/293

(57) ABSTRACT

The present invention relates to a compound of formula (I),

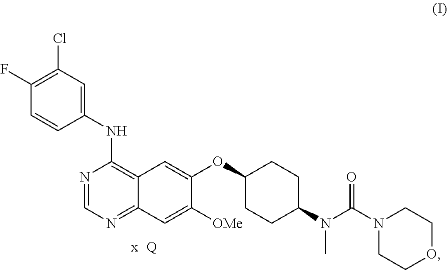

wherein x Q denotes x H₂O x HCl; or x 0.5 HCl x 1.5 H₂O,
   which have valuable pharmacological properties, particularly an inhibitory effect on signal transduction mediated by tyrosine kinases, processes for the stereoselective preparation of this compound, particularly for the inhalation of suitable pharmaceutical formulations and their use for the treatment of diseases, particularly tumour diseases, benign prostatic hyperplasia and diseases of the lungs and airways.

The present invention relates to compounds of formula (I),

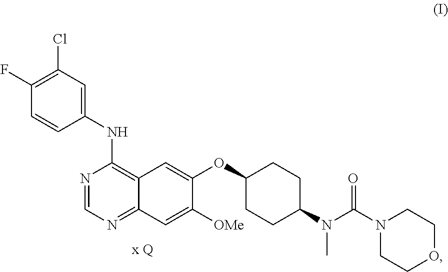

wherein x Q denotes x H₂O x HCl; or x 0.5 HCl x 1.5 H₂O,
   which have valuable pharmacological properties, particularly an inhibitory effect on signal transduction mediated by tyrosine kinases, processes for the stereoselective preparation of these compounds, particularly for the inhalation of suitable pharmaceutical formulations and their use for the treatment of diseases, particularly tumour diseases, benign prostatic hyperplasia and diseases of the lungs and airways.

### BACKGROUND TO THE INVENTION

Quinazoline derivatives are known from the prior art as active substances for example for the treatment of tumour diseases and also diseases of the lungs and airways. Processes for preparing quinazoline derivatives are described in WO03082290 and WO07068552.

The aim of the present invention is to provide salts and hydrates of 4-[(3-chloro-4-fluoro-phenyl)amino]-6-(cis-4-{N-[(morph-1-olin-4-yl)carbonyl]-N-methyl-amino}-cyclohexan-1-yloxy)-7-methoxy-quinazoline which by virtue of their pharmaceutical activity as tyrosine kinase inhibitors are suitable for use in the therapeutic field, i.e. for the treatment of pathophysiological processes caused by hyperfunction of tyrosine kinases.

The compound prepared in the present invention is supposed to meet the requirements for physical and chemical stability and other properties, such as for example sufficient solubility, low hygroscopy and absence of polymorphism, particularly sufficient solubility and absence of polymorphism, as well as in particular low hygroscopy, that are imposed on a pharmaceutical active substance.

Therefore the aim of the present invention is to provide compounds that are suitable particularly for administration by inhalation. The invention also sets out to provide a stereoselective process for preparing the compounds according to the invention as well as a pharmaceutical formulation that is particularly suitable for inhalation, particularly in terms of a suitable particle size distribution of active substance and adjuvants.

### DESCRIPTION OF THE INVENTION

The present invention solves the problems stated above by providing the compounds of formula (I) which are suitable in particular for powder inhalation, which have crystalline stability, low hygroscopy, sufficient solubility and low polymorphism, the pharmaceutical formulation thereof and the method of synthesis described hereinafter.

Compounds suitable for inhalative administration are the compounds of formulae (I.1), (I.2), (I.3) and (6), particularly formula (I.1).

The invention thus relates to compounds of formula (I)

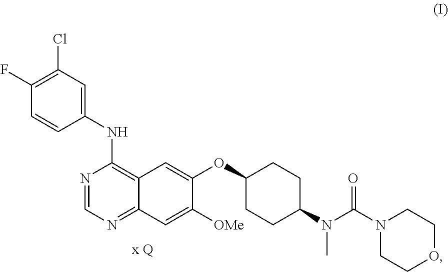

wherein x Q denotes x H₂O x HCl; or x 0.5 HCl x 1.5 H₂O.

Particularly preferred is a compound of formula (I.1)

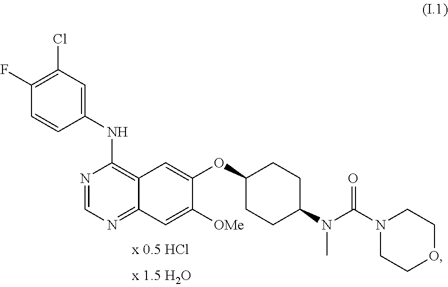

preferably a crystalline compound of formula (I.1), wherein reflections occur in the X-ray powder diagram at dₕₖₗ values [Å] of 16.58 Å, 5.50 Å, 5.30 Å, 4.66 Å, 4.62 Å, 4.24 Å and 3.45 Å.

Compound (I.1) solves the stated problem particularly advantageously, as it does not exhibit any polymorphism and has an advantageous solubility. Moreover, compound (I.1) has hygroscopic characteristics which make it suitable as a stable powder formulation. Depending on the relative humidity, no change in particle size or phase change takes place. Also preferred is a compound of formula (I.2)

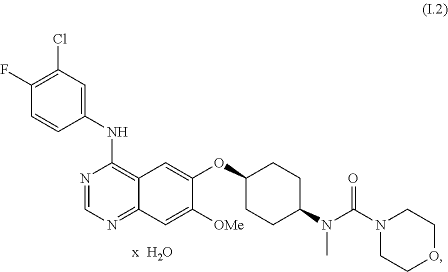

preferably a crystalline compound of formula (I.2), wherein reflections in the X-ray powder diagram occur at dₕₖₗ values of 9.60 Å, 6.32 Å, 5.00 Å, 4.68 Å, 3.63 Å and 3.47 Å.

Also preferred is a compound of formula (I.3)

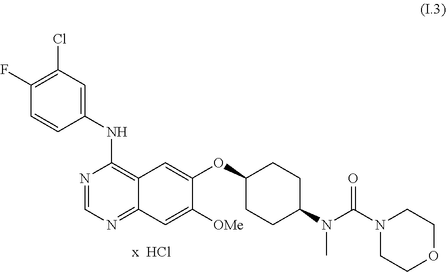

particularly in its low-melting or high-melting form.

Particularly preferred is a crystalline low-melting form of the compound of formula (I.3), in which reflections in the X-ray powder diagram occur at dₕₖₗ values of 21.02 Å, 9.37 Å, 7.10 Å, 4.94 Å, 4.78 Å and 3.44 Å.

Particularly preferred is a crystalline high-melting form of the compound of formula (I.3), in which reflections in the X-ray powder diagram occur at dₕₖₗ values of 18.89 Å, 9.12 Å, 5.86 Å, 3.81 Å and 3.52 Å

The invention further relates to a pharmaceutical composition containing a compound of formula (I), particularly of formula (I.1).

Preferably, a pharmaceutical composition containing a compound of formula (I), particularly of formula (I.1) is in the form of a powder mixture suitable for inhalation. Also preferred is a pharmaceutical composition containing a compound of formula (I), characterised in that it is a mixture of a compound of formula (I), particularly of formula (I.1), with lactose.

Particularly preferred is a pharmaceutical composition containing a compound of formula (I), characterised in that it is a mixture of interactive powder mixture and agglomerated clusters of the compound of formula (I), particularly of formula (I.1), with lactose.

Particularly preferred is a pharmaceutical composition containing a compound of formula (I) (hereinafter referred to as “formulation”), preferably of formula (I.1), which represents a mixture of interactive powder mixture and agglomerated clusters of micronised active substance and fine particles or fine fractions contained in the lactose used. By interactive powder mixture is meant a powder mixture in which each particle of active substance adheres to a coarser carrier adjuvant. The agglomerated clusters described here are from 5 to 200 μm in size, preferably from 10 to 100 μm, and consist of micronised active substance particles and fine particles contained in the lactose used.

The formulation contains an inhalable dose in the range from 2 to 6 mg. With regard to the inhalable dose, the flow rate dependency during the patient's breathing-in maneuver is found to be advantageously low. The flow rate dependency is defined as the quotient of the inhalable dose at a 1 and 4 kPa pressure drop when using the inhaler in question. The inhaler used may be a capsule-based or blister-based powder inhaler.

High-dose powder mixtures are particularly suitable as the formulation. They are not interactive powder mixtures, but constitute a mixture of interactive powder mixture and agglomerated clusters of the micronised active substance. The addition of coarse adjuvant results in an improvement in dispersion compared with pure micronised active substance.

The formulation consists of jet-ground active substance having a particle size distribution with a d₅₀ value of 1 to 5 μm, preferably 2.5 to 5 μm, and a conventional adjuvant such as α-lactose monohydrate, for example. However, any other pharmaceutically inactive adjuvant may be used, selected for example from among low-molecular carbohydrates and/or sugar alcohols, particularly trehalose, mannitol, inositol, isomalt, sorbitol, xylitol, maltitol, erythritol and myo-inositol. The distribution of the particle size of the adjuvant may be mono- or bimodal. The particle size distribution of the adjuvant for monomodal distributions preferably preferably has a d₅₀ value of 2 to 200 μm, preferably 30 to 120 μm. Bimodal distributions of the adjuvant are made up of a fine fraction with a d₅₀ value of 2 to 10 μm, preferably from 2 to 6 μm, and a coarse fraction with a d₅₀ value of 20 to 200 μm, preferably from 40 to 120 μm. Different adjuvants may form the fine and coarse fractions. The active substance concentrations in the powder mixture, for monomodal particle size distributions of the adjuvant, are preferably 5 to 50%, preferably 20 to 40%. In bimodal distributions the active substance concentration is also from 5 to 50%, preferably from 20 to 40%. The two fractions of the bimodal distribution of the adjuvant are in ratio, based on the mass (fine:coarse), of from 3:1 to 1:3, preferably from 1:1 to 1:2. The total amount of the powder mixture is preferably 10 to 40 mg, depending on the dosage required in the lungs.

The high lung-bound fraction based on the amount of active substance weighed in, ranging from 10 to 60%, preferably from 30 to 50%, as well as the low flow rate dependency ranging from 0.5 to 1.0, make it possible to achieve the maximum pharmaceutical performance.

The production example that follows serves to illustrate the process carried out by way of example. It is to be understood as being an illustration of the invention without restricting it to the specific content thereof.

### Pharmaceutical Formulation Examples

### a) Capsules for Powder Inhalation Containing 5 mg Active Substance

1 capsule contains:

| | |
|---|---|
| Active substance | 5.0 mg |
| Lactose for inhalation purposes | 15.0 mg |
| | 20.0 mg |

### b) Capsules for Powder Inhalation Containing 11 mg Active Substance

1 capsule contains:

| | |
|---|---|
| Active substance | 11.0 mg |
| Lactose for inhalation purposes | 24.0 mg |
| | 35.0 mg |

### Preparation of the Formulation

Active substance and adjuvant are each added in portions alternately through a screen and collected in a container in the same way that a lasagne is constructed. The accumulation of powder thus produced is then transferred into a second container, which should not be filled to a level of more than 50% by volume. This container is clamped in a tumbler mixer and mixing is carried out (e.g. 30 min at 30 rpm). It is also possible to use a forcing mixer. The powder mixture thus produced is used to fill capsules for inhalation or blister cavities.

The invention further relates to a pharmaceutical product which in the pharmaceutical composition according to the invention consists of the formulation within a powder inhaler. Inhaler designs that are suitable for the formulation include for example powder inhalers selected from among the HandiHaler, pm-MDPI, reservoir MDPI, unit-dose DPI and disposable unit-dose DPI, preferably the HandiHaler and pm-MDPI.

The invention also relates to the compound of formula (I) described above, particularly the compound of formula (I.1), for use as medicaments, particularly for the treatment of inflammatory or allergic diseases of the airways, preferably for the treatment of COPD and/or chronic bronchitis.

The invention further relates to a process for the stereoselective preparation of a compound of formula (I.1)

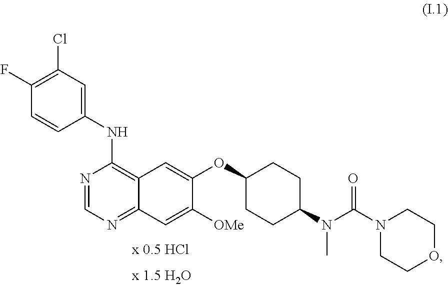

the process comprising reaction steps (A) to (E), wherein
(A) denotes the hydrogenation of a compound of formula (I)

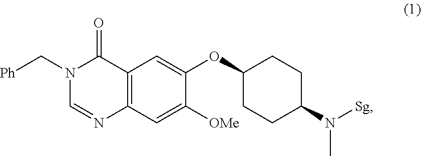

to form a compound of formula (2),

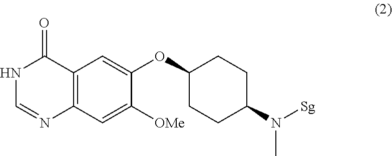

(B) denotes the reaction of a compound of formula (2)

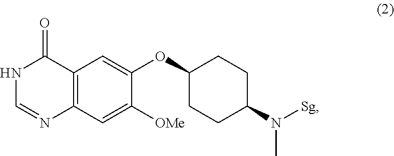

via the intermediate of formula (3)

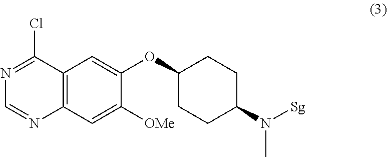

to form the compound of formula (4)

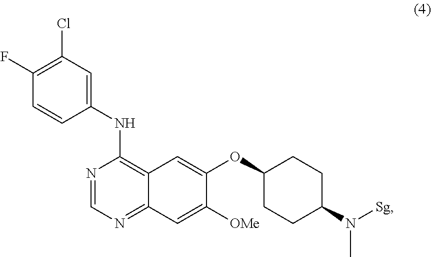

(C) denotes the reaction of the compound of formula (4), optionally after a preceding purification step, to form a compound of formula (5.1) or (5.2)

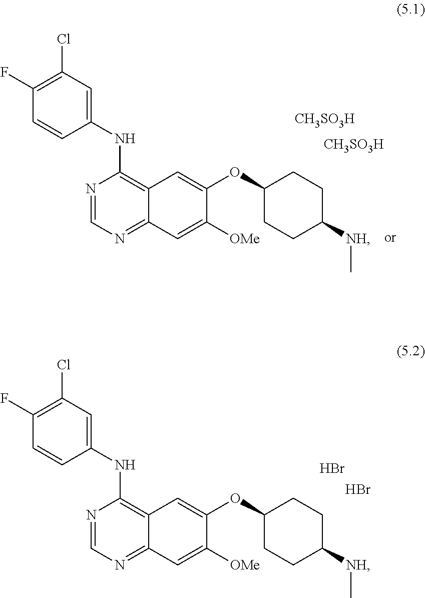

(D) denotes the reaction of the compound of formula (5.1) or (5.2) with morpholine-4-carbonyl chloride, to form a compound of formula (I.2)

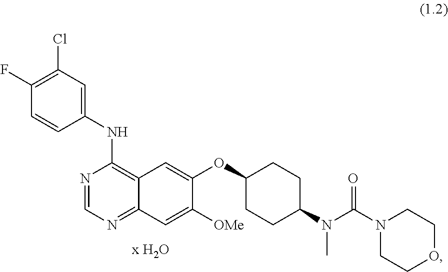

and
(E) denotes the reaction of the compound of formula (I.2) to form a compound of formula (I.1)

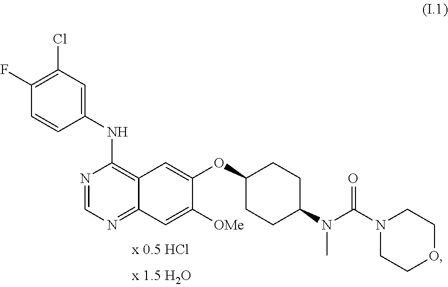

in which the steps (A) to (E) take place successively in the order specified.

In a preferred process for the stereoselective preparation of a compound of formula (I.1), the process consists of step (E).

A particularly preferred process for the stereoselective preparation of a compound of formula (I.1) is characterised in that step (E) is followed by a recrystallisation of the compound of formula (I.1).

Also preferred is a process for the stereoselective preparation of the compound of formula (I.1), in which step (D) is replaced by the successive reaction steps (F) and (G), where
(F) denotes the reaction of the compound of formula (5.1) or (5.2) with bis-[1,2,4]triazol-1-yl-methanone to form a compound of formula (7)

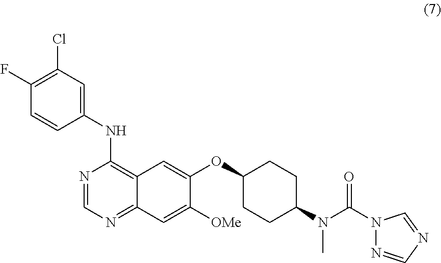

and
(G) denotes the reaction of formula (7) with morpholine to form the compound of formula (I.2).

Particularly preferred is a process for the stereoselective preparation of a compound of formula (I.1), in which the process consists of step (G).

The invention further relates to the intermediate of formula (5.1).

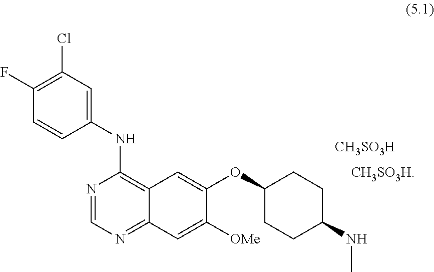

The invention further relates to the intermediate of formula (5.2).

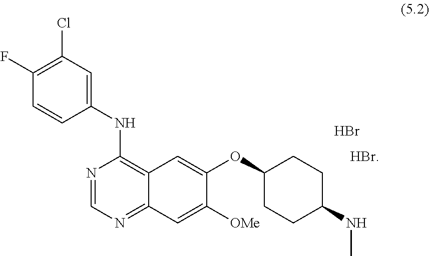

The invention further relates to the crystalline anhydrous form of the compound of formula (6), wherein reflections in the X-ray powder diagram occur at dₕₖₗ values of 5.66 Å, 5.33 Å, 4.89 Å, 4.76 Å, 3.92 Å and 3.31 Å.

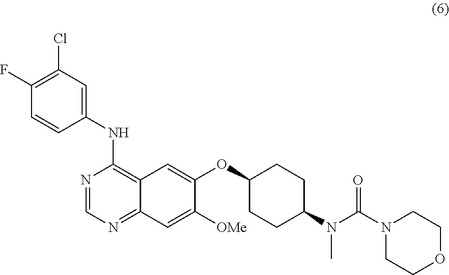

and the pharmacologically acceptable salts, hydrates, solvates and co-crystals thereof. The invention further relates to the compound of formula (I), and the pharmacologically acceptable hydrates, solvates and co-crystals thereof.

By co-crystals are meant, within the scope of the present invention, molecular complexes which contain two or more different molecules in the same crystal lattice (Crystal Growth & Design, 2009, Vol. 9, No. 6, 2950-2967; Stahly, G. P. Cryst. Growth Des. 2007, 7, 1007-1026), particularly co-crystals that are formed between a molecular or ionic pharmaceutical active substance molecule and a co-crystal forming agent that exists as a solid at ambient temperature (Jones, W.; Motherwell, W. D.; Trask, A. V. MRS Bull. 2006, 341, 875-879; Vishweshwar, P.; McMahon, J. A.; Bis, J. A.; Zaworotko, M. J., J. Pharm. Sci. 2006, 95, 499-516).

Within the scope of the present invention the expression compounds with “low hygroscopy” refers to compounds which absorb less than 3% water over the entire humidity range tested (10-90% r.h.) in the water absorption tests described here and which furthermore do not exhibit any phase change induced by the uptake or release of water.

In process steps A catalysts are used which are preferably selected from among Pd/C or Pd(OH)₂, preferably Pd/C.

Also particularly preferred is a process in which in step (B) a chlorinating agent selected from among thionyl chloride, phosphorus oxychloride, an N-chlorosuccinimide/triphenylphosphane, N-chlorophthalimide/triphenylphosphane; 1,3-dichloro-5,5-dimethylhydantoin/triphenylphosphane or trichloroisocynuric acid/triphenylphosphane combination and a carbon tetrachloride/triphenylphosphane combination is used. These are used in the presence of a suitable organic or inorganic base such as for example tertiary amine, e.g. triethylamine, diisopropylethylamine, diethylaniline or inorganic carbonates, inorganic hydrogen carbonates or inorganic phosphates. Also particularly preferred is a process in which step (B) must be carried out in the presence of an organic or inorganic base such as for example tertiary amines, e.g. triethylamine, diisopropylethylamine or diethylaniline, inorganic carbonates, inorganic hydrogen carbonates or inorganic phosphates.

In step (C), HCl may be used, for example, to remove the protective group, particularly the Boc group. Strong acids selected from among sulphuric acid, formic acid, phosphoric acid, p-toluenesulphonic acid (PTSA), phenylsulphonic acid and camphorsulphonic acid are also suitable for this.

In step (D) the reaction may also be carried out in the presence of a suitable base, for example in addition to diisopropylethylamine in the presence of other tertiary amines such as triethylamine or diethylaniline, inorganic carbonates, inorganic hydrogen carbonates or inorganic phosphates.

Other leaving groups, such as, for example, a 1H-imidazole ring, a 1H-1,2,4-triazole, a 1H-1,2,3-triazole or a 1H-benzotriazole ring may replace the chlorine atom in the 4-morpholinocarbonyl chloride.

In step (F) the reaction may be carried out in the presence of a suitable base, for example diisopropylethylamine, other tertiary amines such as triethylamine or diethylaniline, inorganic carbonates, inorganic hydrogen carbonates or inorganic phosphates.

The use of the following solvents selected from the group specified in each case is preferred in the process steps described above:

in step
A: isopropylalcohol, tetrahydrofuran, methyltetrahydrofuran, n-propanol, ethanol, butyl acetate, water or mixtures thereof consisting of two or more components,
B: acetonitrile, dichloromethane, tetrahydrofuran, butyl acetate, toluene and mixtures thereof,
C: isopropylalcohol, methanol, glacial acetic acid, tetrahydrofuran, butyl acetate, toluene and mixtures thereof consisting of two or more components,
D: acetonitrile, dichloromethane, tetrahydrofuran, butyl acetate, toluene and mixtures thereof consisting of two or more components,
E: ethanol, isopropyl alcohol, tetrahydrofuran, methyltetrahydrofuran, n-propanol, water and mixtures thereof consisting of two or more components,
F: tetrahydrofuran, tetrahydrofuran, butyl acetate, toluene, ethanol and mixtures thereof consisting of two or more components,
G: N-methylpyrrolidone, N-ethylpyrrolidone, dimethylformamide, dimethylsulphoxide, toluene and xylene.

The process steps described above are preferably carried out in the following temperature ranges:

in step:
A: preferably 20 to 120° C., particularly preferably 80 to 110° C.,
B1: preferably 20 to 100° C., particularly preferably 30 to 50° C.,
B2: preferably 0 to 80° C., particularly preferably 20 to 50° C.
C: preferably 20 to 100° C., particularly preferably 50 to 90° C.,
D: preferably 0 to 80° C., particularly preferably 20 to 60° C.,
E: preferably 20 to 80° C., particularly preferably 50 to 78° C.,
F: preferably 0 to 60° C., particularly preferably 15 to 35° C.,
G: preferably 40 to 180° C., particularly preferably 100 to 130° C.,

In the process according to the invention, protective groups selected from among trifluoroacetyl, acetyl, benzoyl, Boc, methoxycarbonyl, ethoxycarbonyl and 2,2,2-trichloroethoxycarbonyl, preferably Boc, are preferably used.

The abbreviation Sg used in this text, including the structural formulae, denotes a protective group. The abbreviation Boc denotes tertiary butyl carbamate.

The term “G-water” used in this text denotes “deionised and sterilised water”.

Scheme 1 illustrates the synthesis according to the invention.

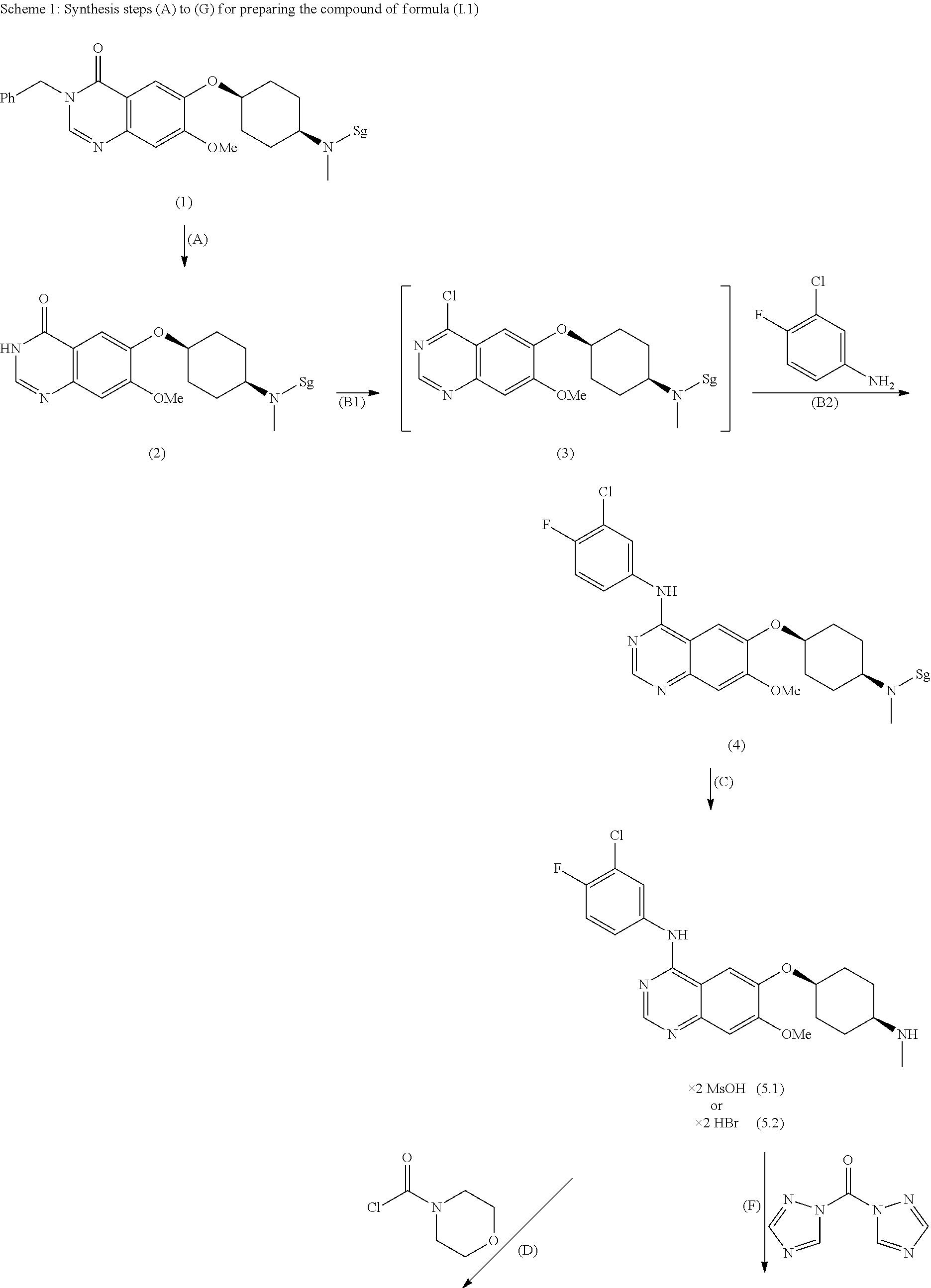

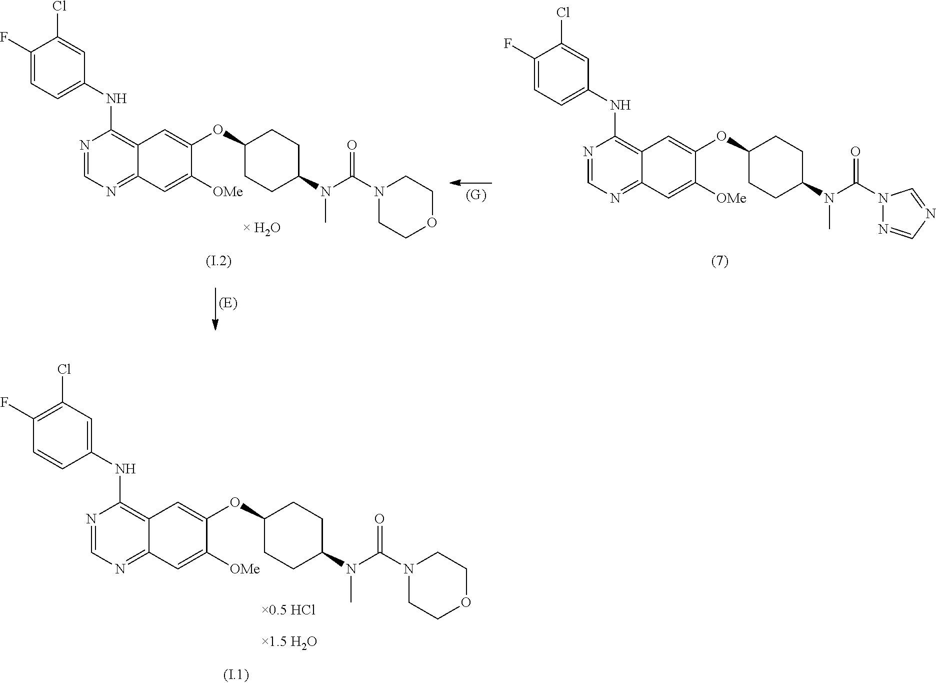

The following Examples serve to illustrate the processes carried out by way of example for preparing the compound of formula (I). These Examples are intended as an illustration of the invention without restricting it to the subject-matter thereof.

### Example 1

### Preparation of Compound (2)—Step (A)

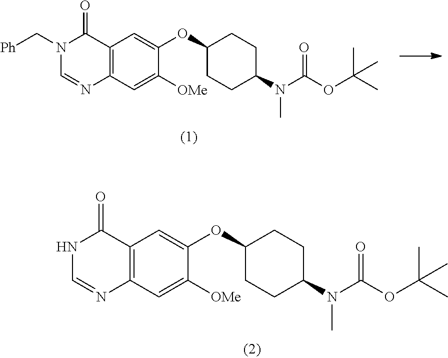

15.00 kg (30.39 mol) of compound (1) are placed in the hydrogenating reactor and 90.0 L isopropyl alcohol are added. 1.50 kg palladium-charcoal (10%, water content 50%) are suspended in 5.0 L of G-water, drawn into the reactor and rinsed with 8.0 L isopropyl alcohol. Then hydrogenation is carried out at 10 bar at an internal temperature of 100° C. until no further uptake of hydrogen can be detected. After the reaction is complete (monitored by HPLC) the catalyst is filtered off and washed with 26.0 L isopropylalcohol.

The hydrogenating solution is placed in a reactor and the container is rinsed with 4.0 L isopropyl alcohol. At normal pressure 75.0 L solvent are distilled off. 150.0 L of n-heptane are added and a further 75.0 L solvent are distilled off (azeotropically) at normal pressure. Then 112.5 l of n-heptane are added, the mixture is cooled to 5° C. and stirred for 30 minutes at 5° C. The product is centrifuged, washed with 30.0 L of n-heptane and dried in the vacuum dryer at 50° C.

m.p. 215° C. (decomposition.)

Mass spectrum (ESI⁺): m/z=m/z 404 [M+H]⁺

TLC Silica gel 60 Rf (CH₂Cl₂/MeOH/NH₄OH 90:10:1)=0.30

¹H-NMR selected data (DMSO-d₆): 12.0 (1H, s), 7.97 (1H, s), 7.50 (1H, s), 7.16 (1H, s), 4.65 (1H, m), 3.92 (3H, s), 2.70 (3H, s).

### Example 2

### Preparation of Compounds (3) and (4)—Step (B)

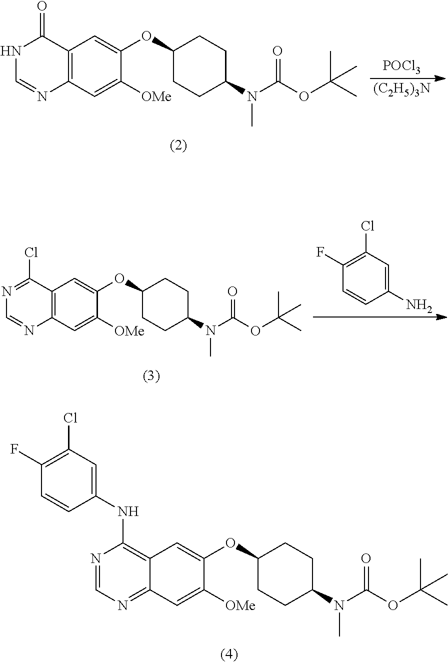

10.00 kg (24.78 mol) of compound (2) are placed in 40 litres of acetonitrile. Then 4.75 kg (30.98 mol) of phosphorus oxychloride are added and the feed vessel is rinsed out with 5 litres of acetonitrile. The suspension is heated to 40° C. and 3.13 kg (30.98 mol) triethylamine are metered in under anhydrous conditions. The feed vessel is rinsed out with 5 L of acetonitrile and stirred for 1 hour at 40° C. The resulting solution is cooled to 20° C. and at this temperature a solution of 4.87 kg (33.46 mol) 3-chloro-4-fluoroaniline in 15 litres of acetonitrile is metered in. The mixture formed is rinsed with 5 litres of acetonitrile, the suspension is heated to 40° C. and stirred for 60 minutes. The reaction mixture is metered at 40° C. onto a mixture of 30 L acetonitrile, 8.44 kg (123.92 mol) industrial-grade ammonia (25%) and 40 litres of G-water. This is then rinsed with 10 L acetonitrile. The suspension is diluted with 20 litres of G-water, cooled to 30° C. and stirred for 30 minutes. The product is centrifuged off and washed with a mixture of 10 litres of G-water and 20 litres of acetonitrile. The product is dried at 50° C. in the drying cupboard.

### Purification of Compound (4):

a) Extraction from Acetonitrile

10.00 kg (18.8 mol) of compound (4) are placed in 50 litres of acetonitrile. The suspension is heated to 80° C. and stirred for 30 min. at this temperature. It is cooled to 50° C., stirred for 30 min. at this temperature and centrifuged. The product in the centrifuge is washed with 20 L of acetonitrile.

b) Extraction from G-Water

The moist product from step a) is suspended in 50 L of G-water. Then it is heated to 80° C. and stirred for 30 min. at this temperature. Then it is cooled to 50° C., stirred for 15 min. at this temperature and centrifuged. The product is washed in the centrifuge with 40 L of G-water. It is dried at 50° C. in the drying cupboard.

m.p. 227° C. (decomposition)

Mass spectrum (ESI⁺): m/z=m/z 531-533 [M+H]⁺

TLC Silica gel 60 Rf (CH₂Cl₂/MeOH 90:10): 0.65.

¹H-NMR selected data (DMSO-d₆): 9.96 (1H, s), 8.13 (1H, dd, J=7.4 and 2.5 Hz), 8.10 (1H, s), 7.80 (1H, m), 7.45 (1H, t, J=9.1), 7.25 (1H, s), 4.86 (1H, m), 3.97 (3H, s), 2.72 (3H, s).

### Example 3

### Preparation of the Compound (5.1)—Step (C)

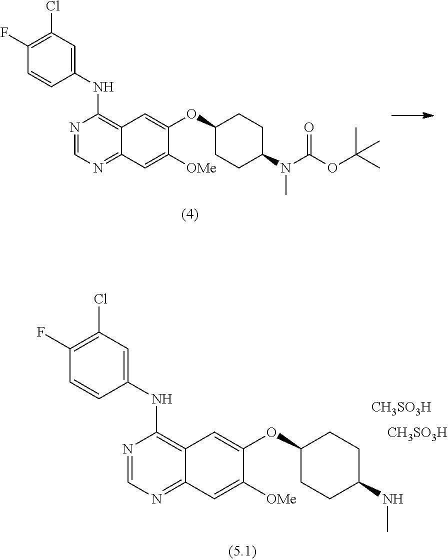

10.00 kg (18.83 mol) of compound (4) are placed in 140 litres isopropyl alcohol. The suspension is heated to 70° C. and 9.05 kg (94.16 mol) methanesulphonic acid are metered in. The feed vessel is rinsed out with 10 L isopropylalcohol and the reaction mixture is heated to reflux temperature. After at least 30 minutes stirring at reflux temperature and total reaction (monitored by HPLC) the suspension is cooled to 20° C. The suspension is then stirred for 30 minutes. The product is centrifuged off and washed with a total of 40 litres of isopropyl alcohol. It is dried at 60° C. in the drying cupboard.

m.p. 282° C. (decomposition)

Mass spectrum (ESI⁺): m/z=431-433 [M+H]⁺

TLC Silica gel 60 Rf (CH₂Cl₂/MeOH/NH₄OH 90:10:1): 0.14.

¹H-NMR selected data: 11.00 (1H, s), 8.90 (1H, s), 8.50 (2H, s), 8.14 (1H, s), 7.98 (1H, dd, J=6.7 and 2.5 Hz), 7.70 (1H, m), 7.56 (1H, t, J=9.2), 7.43 (1H, s), 4.89 (1H, m), 4.02 (3H, s), 2.40 (6H, s).

### Preparation of Compound (5.2)—Step (C)

Alternatively compound (5) may be isolated as the dihydrobromide (compound (5.2)) using hydrogen bromide.

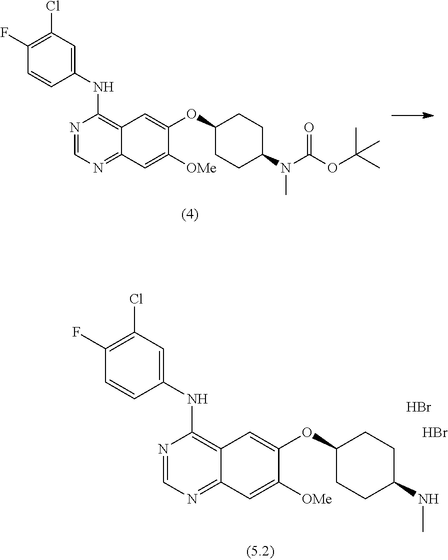

1.0 g (1.88 mmol) of compound (4) are placed in 14 mL glacial acetic acid. 1.34 mL (7.42 mmol) of hydrogen bromide 33% in glacial acetic acid is added, the suspension is heated to 74° C. and after 1.5 hours the suspension is cooled to 20° C. 22 mL ethyl acetate is added. The suspension is then stirred for 2 hours. The product suspension was suction filtered, washed with tert.butylmethyl ether and dried at 45° C. in the vacuum drying cupboard.

¹H-NMR selected data: 11.30 (1H, s), 8.92 (1H, s), 8.63 (2H, s), 8.43 (1H, s), 8.01 (1H, dd, J=6.4 and 2.6 Hz), 7.75 (1H, m), 7.56 (1H, t, J=8.8), 7.43 (1H, s), 5.03 (1H, m), 4.03 (3H, s).

### Example 4

### Preparation of Compound (I.2)—Step (D)

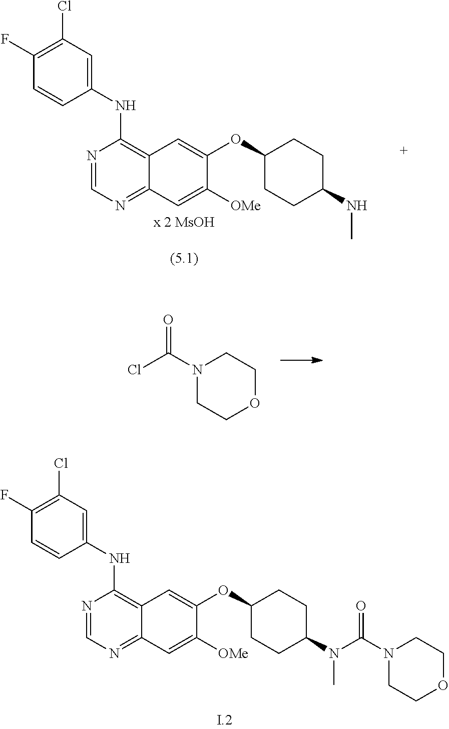

12.00 kg (19.26 mol) of compound (5.1) are suspended in 60.0 L acetonitrile. 10.45 kg (80.88 mol) diisopropylethylamine are added and the mixture is rinsed with 6.0 L acetonitrile. The reaction mixture is heated to 50° C. and 4.32 kg (28.89 mol) of 4-morpholinocarbonyl chloride are added at 50° C. The mixture is rinsed with 6.0 L acetonitrile. The reaction mixture is stirred for 2-3 hours at 50° C. After the reaction is complete (monitored by HPLC) the mixture is added to 120.0 L of G-water and then the product temperature is adjusted to 25° C. The suspension is stirred for 30 minutes at 25° C. The product is filtered off, washed with 2×36.0 L of G-water and dried with entraining gas at 70° C. in the filter dryer in vacuo.

m.p. 247° C.

Mass spectrum (ESI⁺): m/z=544-546 [M+H]⁺

TLC Silica gel 60 Rf (CH₂Cl₂/MeOH/NH₄OH 90:10:1): 0.64.

¹H-NMR selected data (DMSO-d₆): 9.50 (1H, s), 8.50 (1H, s), 8.11 (1H, dd, J=6.7 and 2.6 Hz), 7.93 (1H, s), 7.79 (1H, m), 7.44 (1H, t, J=9.5 Hz), 7.24 (1H, s), 4.80 (1H, m), 3.96 (3H, s), 3.71 (1H, m), 3.60 (4H, br t, J=4.2 Hz), 3.08 (4H, br t, J=4.2 Hz).

### Example 5

### Preparation of Compound (I.1)—Step (E)

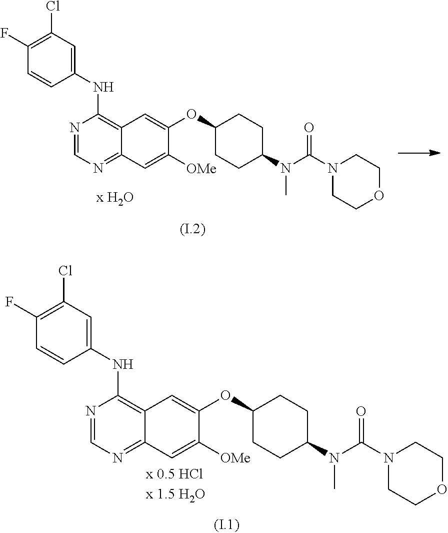

10.00 kg (18.38 mol) of compound (I.2) are suspended in 95.0 L of absolute ethanol and heated to 75° C. At 75° C., 8.66 kg (9.26 mol) hydrochloric acid (3.90%) are added. The feed vessel is rinsed with 2.5 L of G-water and 5.0 L of absolute ethanol. The reactor contents are refluxed and stirred until a solution is obtained. The solution is cooled to 70° C., filtered clear through a pressure filter into a second reactor, rinsed with 20.0 L absolute ethanol, and 60.0 L of solvent are distilled off in vacuo. The reactor contents are cooled to 22° C. and the suspension is stirred for 30 minutes at 22° C. The product (compound (I.1)) is filtered off, washed with 2×15.0 L of G-water and dried in vacuo at 60° C. with entraining gas in the filter dryer.

Optionally the product may be recrystallised as described hereinafter:

10.00 kg (17.78 mol) of compound (I.1) is suspended in 72.0 L of absolute ethanol and 18 L of G-water. The reactor contents are refluxed and stirred until a solution is obtained. The solution is cooled to 70° C., filtered clear through a pressure filter into a second reactor and rinsed with a mixture of 16.0 L of absolute ethanol BPH and 4 L of G-water. The reactor contents are cooled to 22° C. and the suspension is stirred for 30 minutes at 22° C. The product is filtered off, washed with 2×20.0 L of G-water and dried in vacuo at 60° C. with entraining gas in the filter dryer.

¹H-NMR selected data (DMSO-d₆): 10.7 (1H, br s), 8.68 (1H, s), 8.28 (1H, s), 8.10 (1H, dd, J=6.7 and 2.6 Hz), 7.80 (1H, m), 7.48 (1H, t, J=9.2 Hz), 7.31 (1H, s), 4.93 (1H, m), 3.99 (3H, s), 3.70 (1H, m), 3.59 (4H, br t, J=4.4 Hz), 3.08 (4H, br t, J=4.4 Hz).

Mass spectrum (ESI⁺): m/z=544-546 [M+H]⁺

TLC Silica gel 60 Rf (CH₂Cl₂/MeOH/NH₄OH 90:10:1): 0.64.

### Example 6

### Preparation of Compound (7)—Step (F)

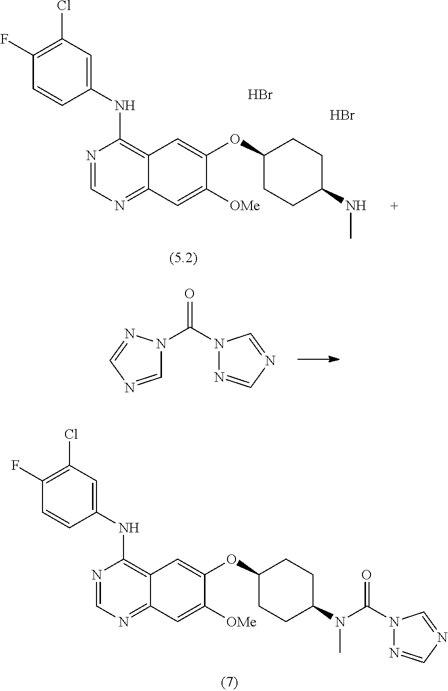

10 g (content approx. 90%, 15.2 mmol) of compound (5.2) are suspended in 100 mL THF (tetrahydrofuran) and 6.5 mL diisopropylethylamine (38.0 mmol). 1,1′-carbonyldi(1,2,4-triazole) (3.95 g, 25.3 mmol) is added at RT. After 20 hours, further 1,1′-carbonyldi(1,2,4-triazole) (0.13 g, 0.79 mmol) is added. After 20 min, 100 mL water are added dropwise. The reaction mixture is stirred for 4 hours at RT and then cooled to 5° C.

The precipitate is suction filtered and washed twice with 10 mL of a 1:1 mixture of water:THF. The product (compound (7)) is dried at 50° C. in the circulating air dryer.

Mass spectrum (ESI⁺): m/z=526-528 [M+H]⁺

¹H-NMR selected data (DMSO-d₆): 9.50 (1H, s), 9.06 (1H, s), 8.51 (1H, s), 8.24 (1H, s), 8.12 (1H, dd, J=7.1 and 2.5 Hz), 7.96 (1H, s), 7.78 (1H, m), 7.44 (1H, t, J=8.98 Hz), 7.25 (1H, s). 4.82 (1H, m), 3.98 (3H, s),

### Example 7

### Preparation of Compound (I.2)—Step (G)

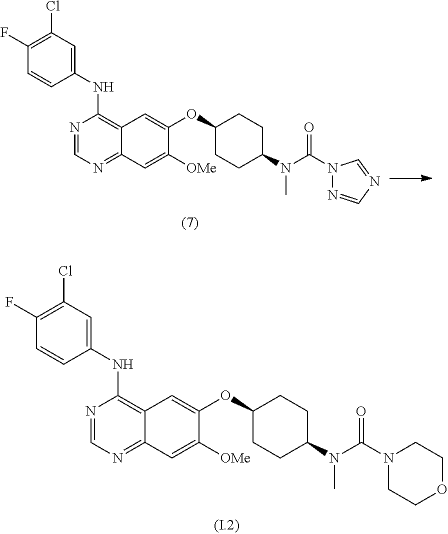

24 g of compound (7) (45.6 mmol) are placed in 72 mL N-methylpyrrolidone and mixed with 12 mL of morpholine (137.7 mmol). The resulting yellow solution is stirred at 120° C. After 5 hours at this temperature the reaction solution is cooled to 90-80° C. Water (360 mL) is slowly added dropwise. After the addition of another 20 mL water, crystallisation sets in at 78° C. The suspension is stirred overnight at RT. The precipitate is suction filtered and washed three times with 20 mL water. The product is dried in the circulating air dryer at 50° C.

Mass spectrum (ESI⁺): m/z=544-546 [M+H]⁺

¹H-NMR selected data (DMSO-d₆): 9.50 (1H, s), 8.50 (1H, s), 8.11 (1H, dd, J=6.7 and 2.6 Hz), 7.93 (1H, s), 7.79 (1H, m), 7.44 (1H, t, J=9.5 Hz), 7.24 (1H, s), 4.80 (1H, m), 3.96 (3H, s), 3.71 (1H, m), 3.60 (4H, br t, J=4.2 Hz), 3.08 (4H, br t, J=4.2 Hz).

TLC Silica gel 60 Rf (CH₂Cl₂/MeOH/NH₄OH 90:10:1): 0.64

### Example 8

### Preparation of Compounds (I.2) and (6)

(3-Chloro-4-fluoro-phenyl)-[7-methoxy-6-(4-methylamino-cyclohexyloxy)-quinazolin-4-yl]-amine dihydrochloride (20.2 g, 40.0 mmol) is suspended in a mixture of acetonitrile (180 mL) and N,N-diisopropylamine (29.0 ml, 166.4 mmol) and at 5° C. treated with a solution of morpholinocarbonyl chloride (6.8 ml, 58.3 mmol) in acetonitrile (20 ml) for 10 minutes. The resulting mixture is slowly heated to ambient temperature, stirred overnight and finally treated with a 0.1 mol/l aqueous sodium hydroxide solution (50 ml). Water (200 ml) is slowly added to the heterogeneous reaction mixture and after 1 hour's stirring the solid is filtered off. The resulting filter cake is washed with water (2×50 ml). The crude product is dissolved at reflux temperature in a mixture of water (30 ml) and ethanol (330 ml). The solution is filtered hot and then slowly cooled to ambient temperature before finally being cooled to 10° C. The crystallised product (I.2) is filtered off and washed with a 1:1 mixture of ethanol/water (20 ml) and finally dried in vacuo at 50° C.

The product thus obtained (I.2) is heated to 140° C. and baked for about 5 minutes at this temperature to form the anhydrous product (6).

Alternatively the anhydrous form (6) may be obtained by recrystallising the compound (I.2) from dry isopropanol.

### Example 9

### Preparation of Compound (I.1)

Compound (I.2) (1.03 kg, 1.83 mol) is suspended in ethanol (5.76 l) and the resulting mixture is heated to 75-76° C. At boiling temperature water (0.5 l) is added followed by a 1.0 mol/l aqueous hydrochloric acid solution (0.91 l, 0.91 mol). The resulting solution is then cooled to 18° C. and kept there for 5 hours in order to achieve total crystallisation. The crystallised product was filtered off and washed with an 8:2 mixture of ethanol/water (1 l) and finally dried at 50° C. in vacuo.

### Example 10

### Preparation of the Low-Melting Form of the Compound (I.3)

150 mg of compound (I.1) are dissolved in 35 ml of ethanol with heating. The resulting solution is slowly cooled to ambient temperature until crystallisation is observed. The resulting suspension is then stored overnight in the refrigerator at 4° C. The crystals obtained (lower-melting form of compound (I.3)) are in turn filtered off and dried overnight under ambient conditions in the extractor hood.

### Example 11

### Preparation of the High-Melting Form of the Compound (I.3)

Compound (I.2) (32.6 g, 60.0 mmol) is suspended in ethanol (210 ml) and the resulting mixture is heated to 75-76° C. Then a 1.0 mol/l aqueous hydrochloric acid solution (60.0 ml, 60 mmol) is added. The resulting solution is slowly cooled to ambient temperature. The crystallised product (high-melting form of compound (I.3)) is then filtered off and washed with an 8:2 mixture of ethanol/water (2×30 ml) and then dried in vacuo at 50° C.

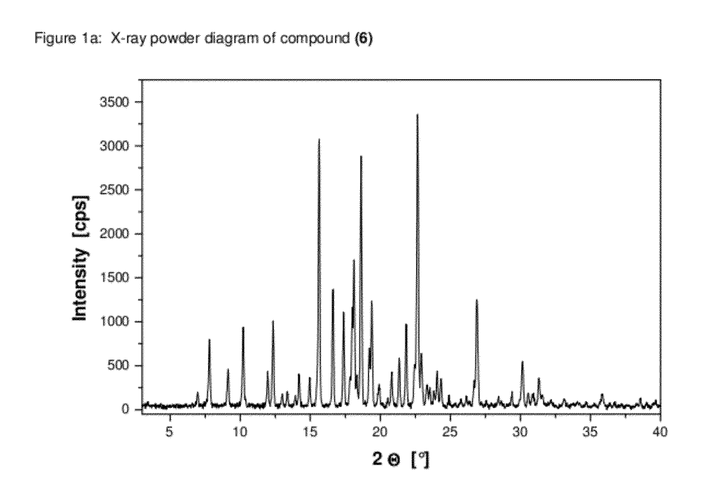

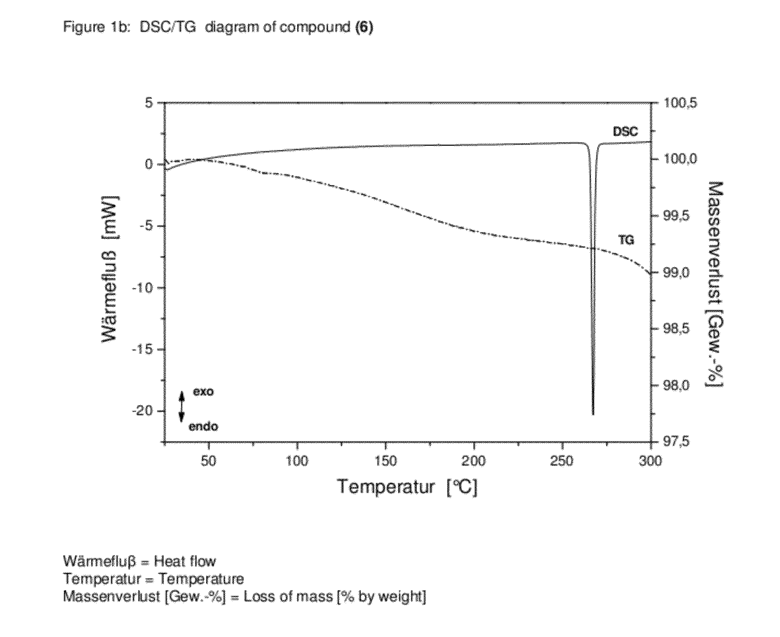

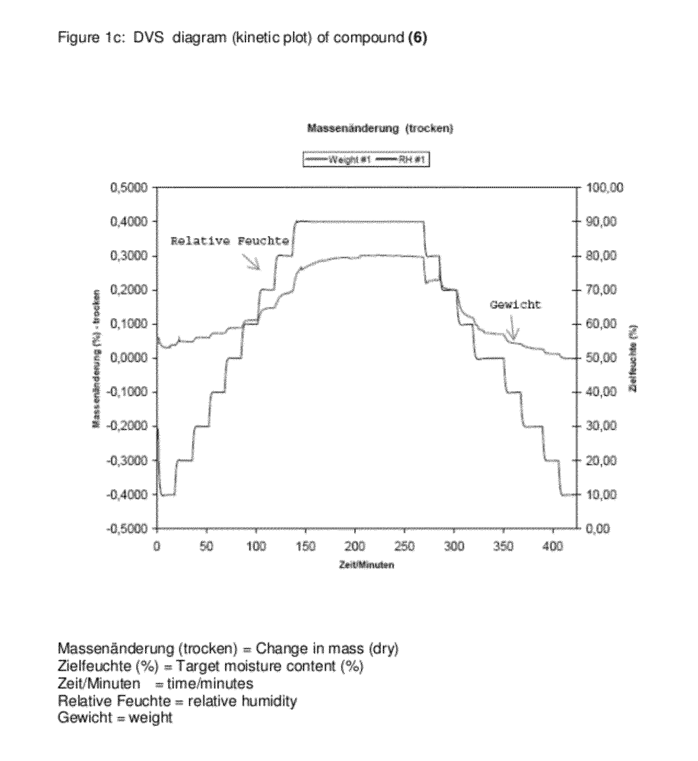

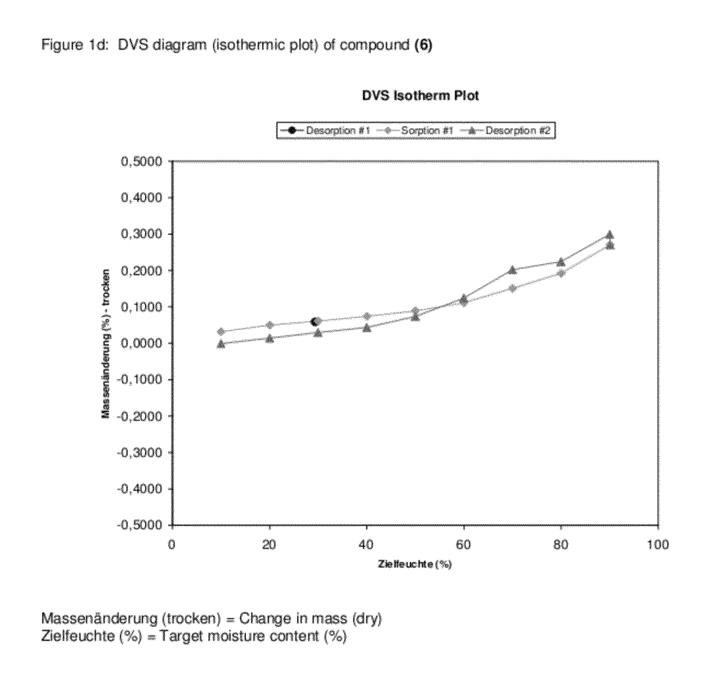

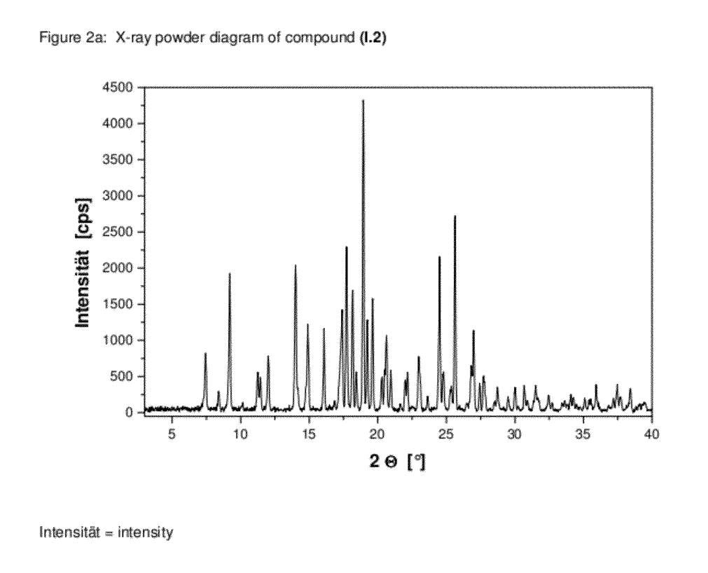

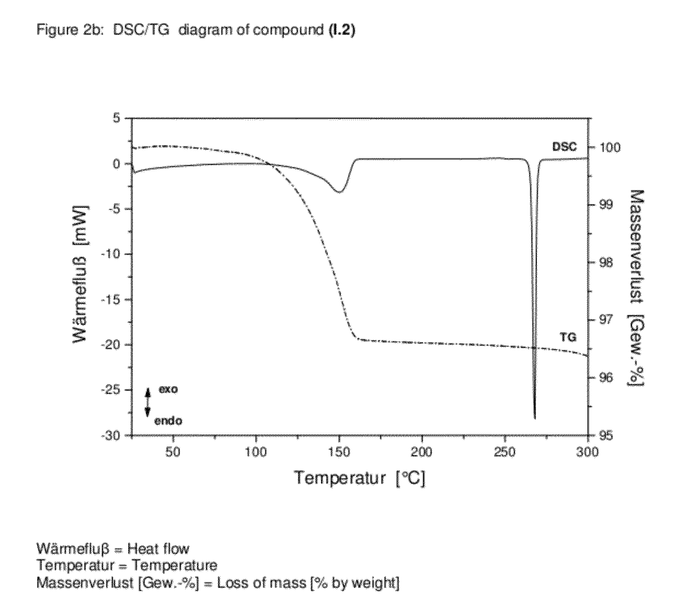

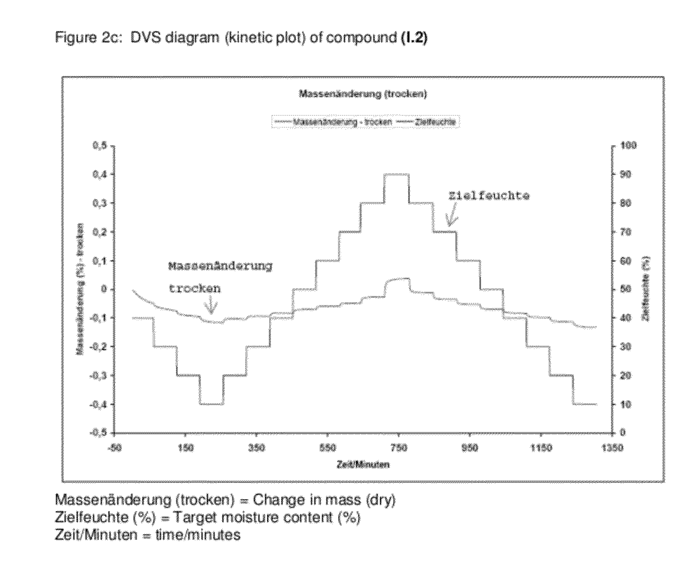

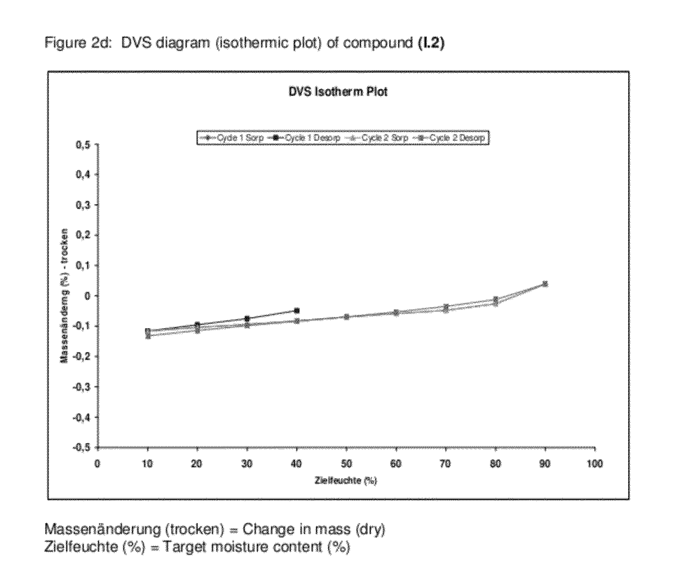

Tables 1 to 6 and FIGS. 1a-1d, 2a-c, 3a-d, 4a-d, 5a-d and 6a-d show further characterising data relating to the compounds according to the invention:

Table 1 Powder X-ray peaks (up to 30° 2Θ) including normalised intensities of the anhydrous form of compound (6)

Table 2: Indexed powder X-ray peaks (up to 30° 2Θ) including normalised intensities of compound (I.2)

Table 3: Powder X-ray peaks (up to 30° 2Θ) including normalised intensities of compound (I.1)

Table 4: Powder X-ray peaks (up to 30° 2Θ) including normalised intensities of the low-melting form of compound (I.3)

Table 5: Powder X-ray peaks (up to 30° 2Θ) including normalised intensities of the high-melting form of compound (I.3)

Table 6: Solubility of the different crystalline forms of compound (I) and compound (6) in water

FIG. 1a: X-ray powder diagram of compound (6)

FIG. 1b: DSC/TG diagram of compound (6)

FIG. 1c: DVS diagram (kinetic plot) of compound (6)

FIG. 1d: DVS diagram (isothermic plot) of compound (6)

FIG. 2a: X-ray powder diagram of compound (I.2)

FIG. 2b: DSC/TG diagram of compound (I.2)

FIG. 2c: DVS diagram (kinetic plot) of compound (I.2)

FIG. 2d: DVS diagram (isothermic plot) of compound (I.2)

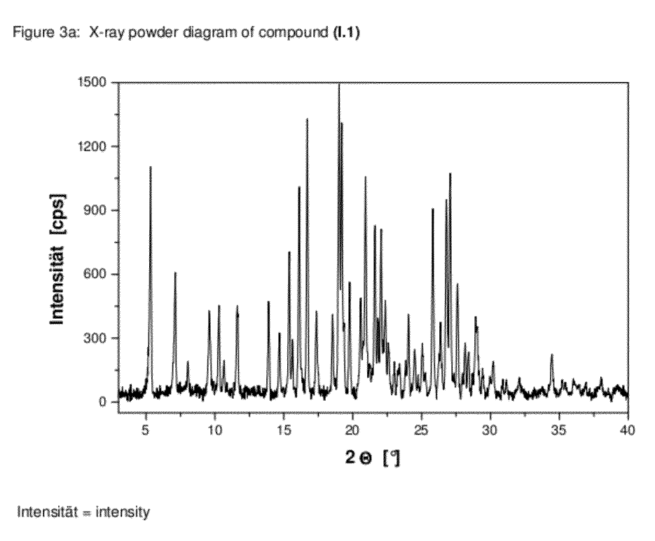

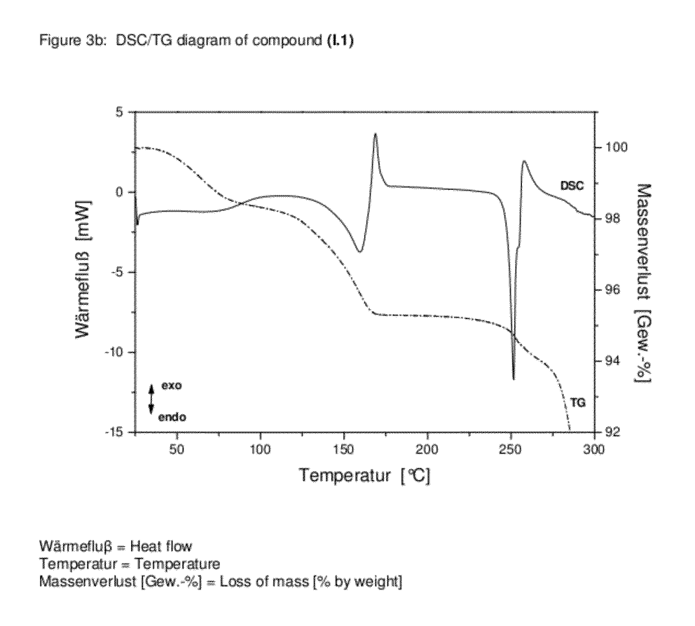

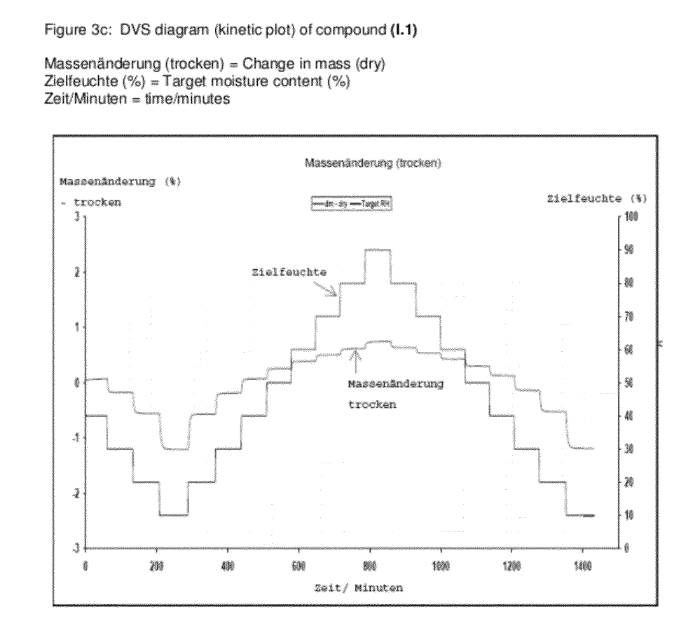

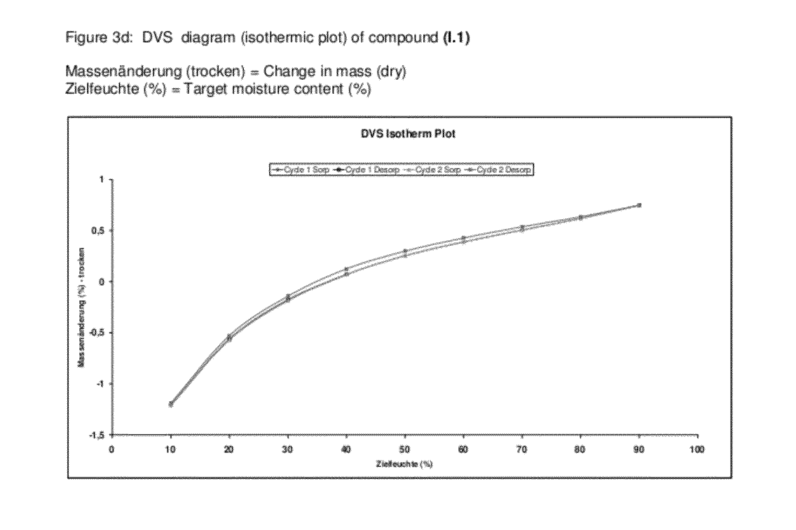

FIG. 3a: X-ray powder diagram of compound (I.1)

FIG. 3b: DSC/TG diagram of compound (I.1)

FIG. 3c: DVS diagram (kinetic plot) of compound (I.1)

FIG. 3d: DVS diagram (isothermic plot) of compound (I.1)

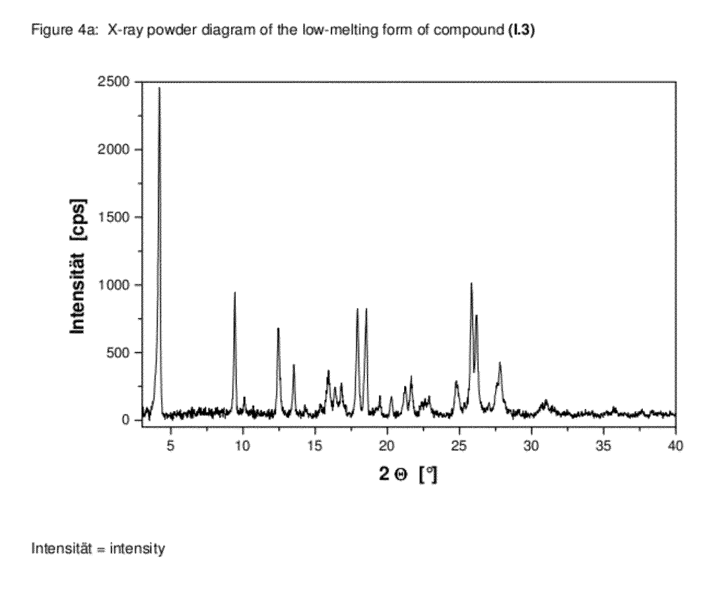

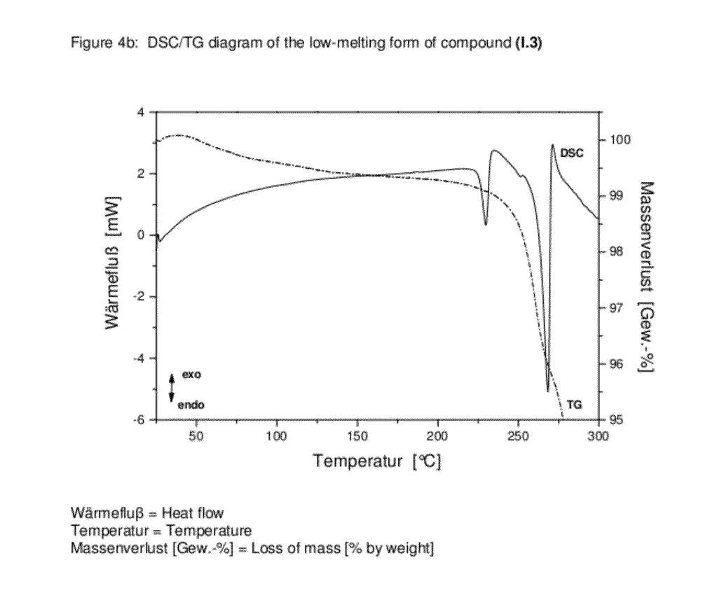

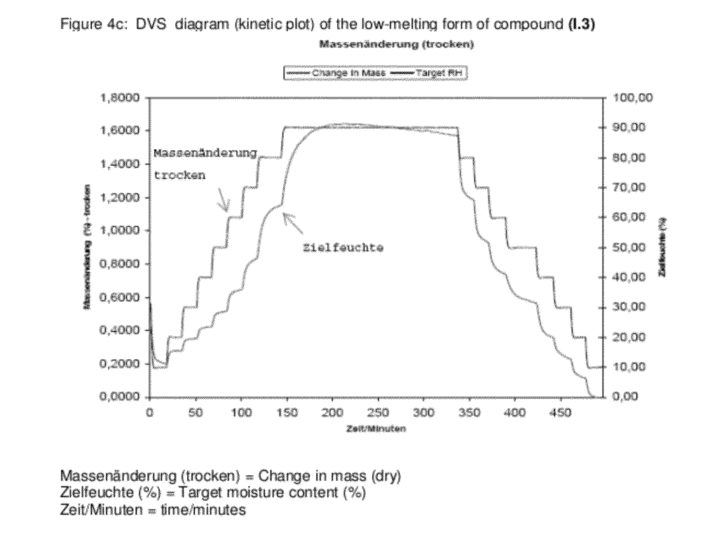

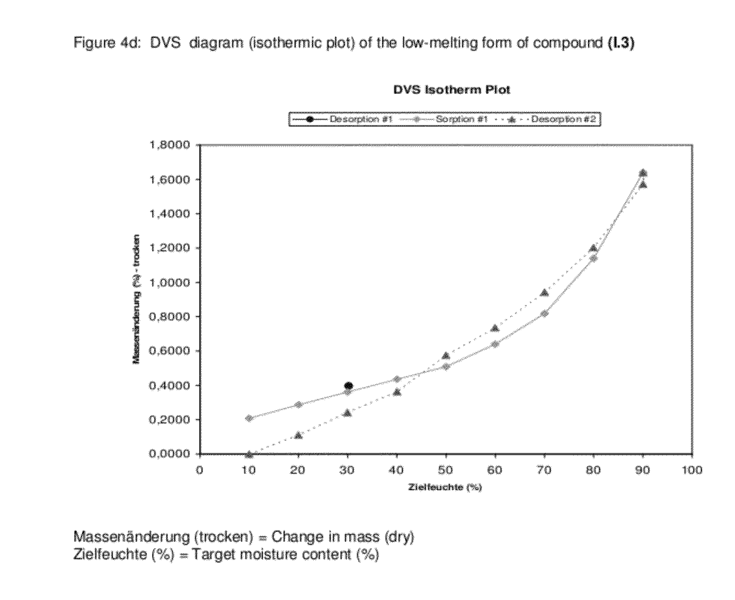

FIG. 4a: X-ray powder diagram of the low-melting form of compound (I.3)

FIG. 4b: DSC/TG diagram of the low-melting form of compound (I.3)

FIG. 4c: DVS diagram (kinetic plot) of the low-melting form of compound (I.3)

FIG. 4d: DVS diagram (isothermic plot) of the low-melting form of compound (I.3)

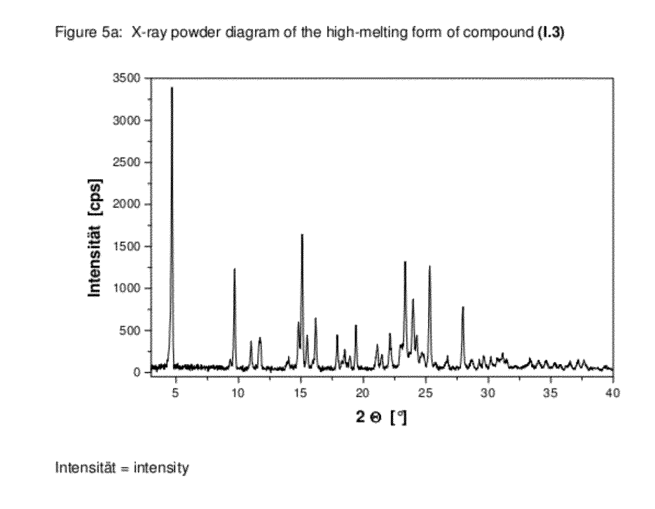

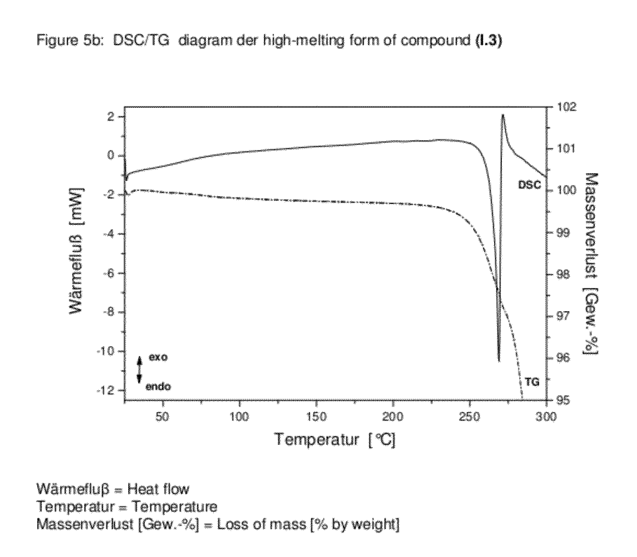

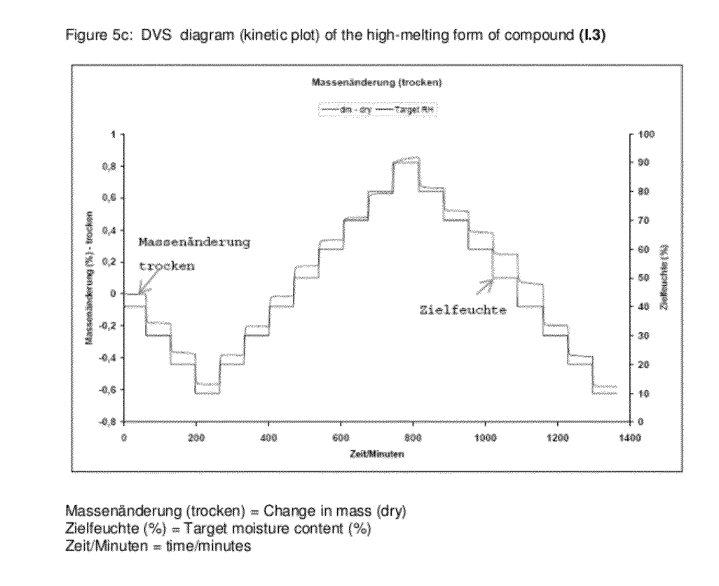

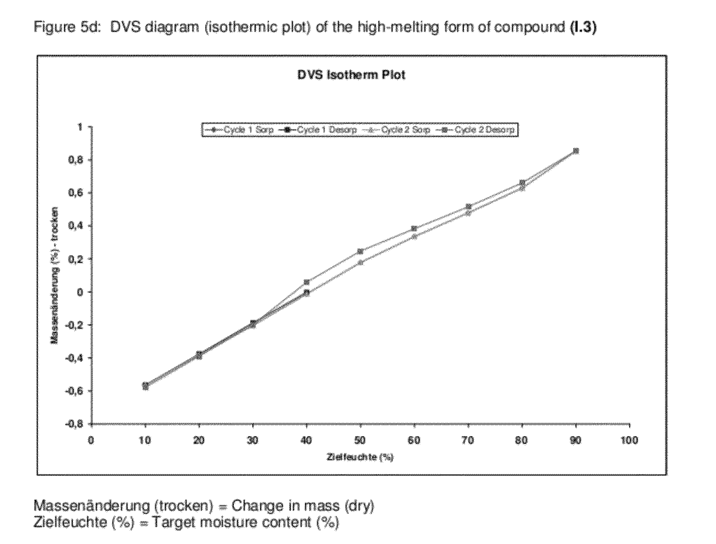

FIG. 5a: X-ray powder diagram of the high-melting form of compound (I.3)

FIG. 5b: DSC/TG diagram of the high-melting form of compound (I.3)

FIG. 5c: DVS diagram (kinetic plot) of the high-melting form of compound (I.3)

FIG. 5d: DVS diagram (isothermic plot) of the high-melting form of compound (I.3)

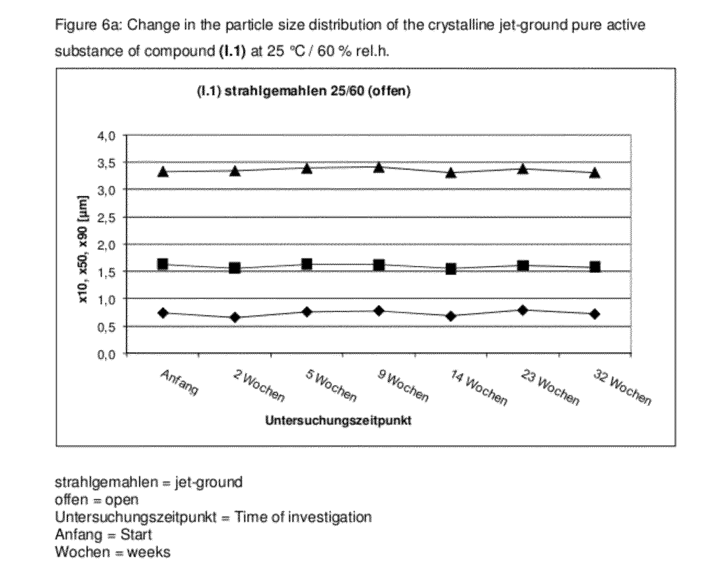

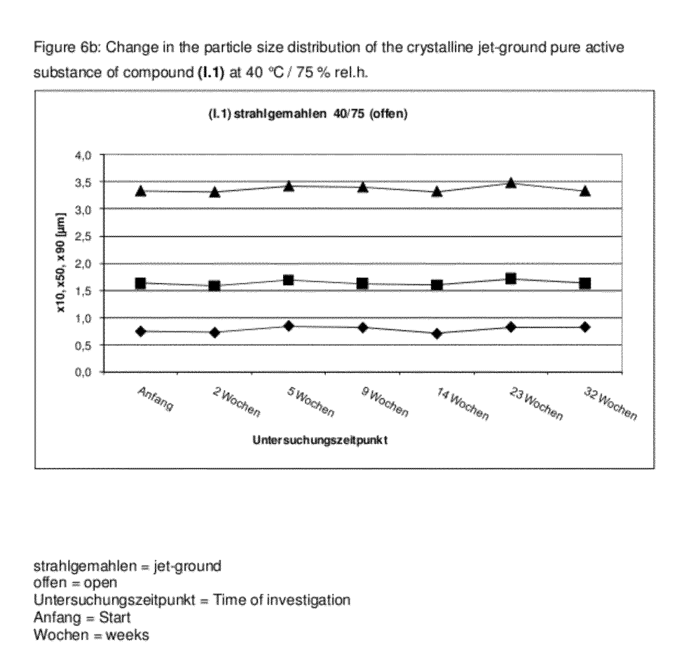

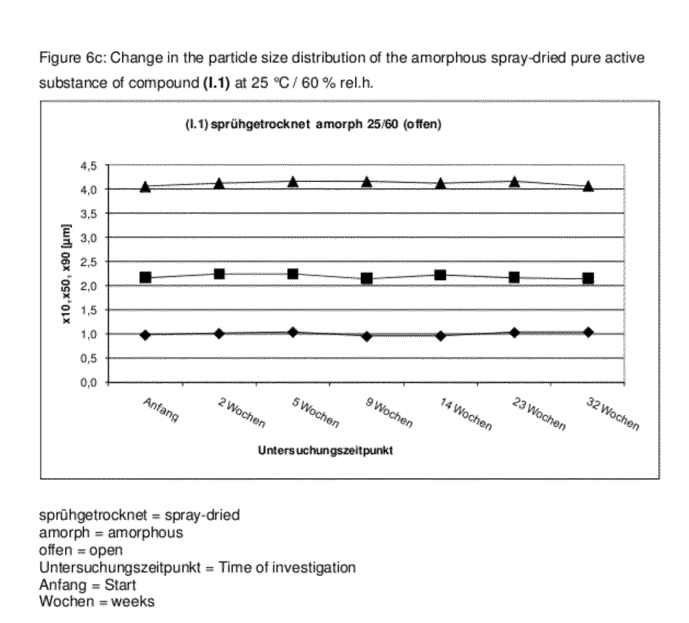

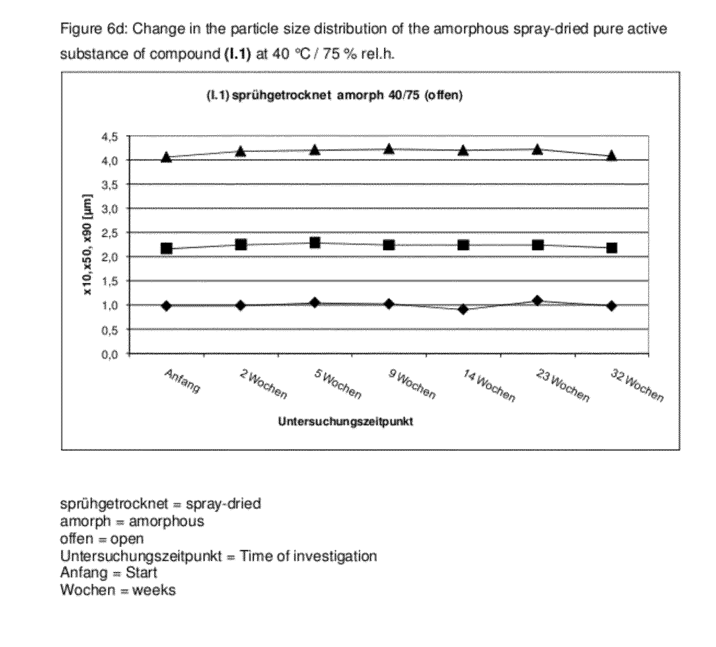

FIG. 6a: Change in the particle size distribution of the crystalline jet-ground pure active substance of compound (I.1) at 25° C./60% rel. h.

FIG. 6b: Change in the particle size distribution of the crystalline jet-ground pure active substance of compound (I.1) at 40° C./75% rel. h.

FIG. 6c: Change in the particle size distribution of the amorphous spray-dried pure active substance of compound (I.1) at 25° C./60% rel. h.

FIG. 6d: Change in the particle size distribution of the amorphous spray-dried pure active substance of compound (I.1) at 40° C./75% rel. h.

The following apparatus and test conditions are used to obtain the data presented in the Appendix:

### X-Ray Powder Diffractometer

STOE Stadi P X-ray powder diffractometer with a location-sensitive detector in transmission mode with a curved germanium (111) primary monochromator; wavelength used: CuK_{αl} with λ=1.540598 Å; X-ray tube operated at 40 kV, 40 mA; 2Θ range: 3-40°

The TREOR programme (which is part of the STOE Stadi P software package) is used to index the X-ray powder diagrams if monocrystal data were available. Tables 2-5 list the characteristic and normalised X-ray reflections up to 30° in 2Θ (±0.5 degrees 2θ). The associated X-ray powder diagrams are shown in FIGS. 2a-5a in the Appendix.

The X-ray powder diagram of compound (I.2) is indexed with the following parameters: monoclinic cell (spatial group P2₁/c) with the following lattice constants:

a=11.445(3) Å, b=23.737(7) Å, c=11.080(3) Å, α=90°, β=113.16(2)°, γ=90°, V=2768(1) Å³.

The “figure of merit” of the indication is 55.3.

### Thermoanalysis Equipment

A DSC 822 made by Mettler Toledo is used. The following measuring parameters are used: heating rate: 10 K/min; type of crucible: perforated aluminium crucible; atmosphere: N₂, 80 ml/min flow rate; typical weights: 3-10 mg.

A TGA/SDTA 851 made by Mettler Toledo, which is coupled to a Nicolet FT-IR 4700 spectrometer (for analysing the volatile fractions), is used. The following measuring parameters are used: heating rate: 10 K/min; type of crucible: open aluminium oxide crucible; atmosphere: N₂, 20 ml/min flow rate; typical weights: 15-25 mg.

The melting points of the different forms may be found in the DSC/TG diagrams (FIGS. 1b-5b) in the Appendix.

### Equipment for Water Absorption Tests

A DVS-1 made by Surface Measurement Systems (=SMS) is used to investigate the hygroscopic characteristics: the following humidity profiles are used: from 10-90% r.h. in steps of 10%, absorption and desorption profiles are recorded, typical weights: 10-20 mg The corresponding diagrams (kinetic and isothermic plot) of the different forms are shown in FIGS. 1c+d-5c+d.

### Solubility Tests

The solubility of the different crystalline forms of formula (I) or (6) in water is determined as follows: approx. 5 mg of the corresponding form are added to 5 ml of water. The mixture is shaken for 2 hours at ambient temperature in an overhead shaker made by the Heidolph company. Then the undissolved fractions are filtered off through a 0.45 μm PTFE filter. The dissolved fraction in the filtrate is determined by UV spectroscopy. In addition, the pH of the saturated aqueous solution is also measured with a standard pH electrode.

The corresponding data are listed in Table 6.

### Stability Tests on Jet-Ground and Spray-Dried Active Substance

Open storage of the crystalline jet-ground and amorphous spray-dried pure active substance under normal climatic conditions (25° C./60% rel. h.) and under extreme climatic conditions (40° C./75% rel. h.) over a period of at least 32 weeks showed no significant changes to the geometric particle size distribution (d₁₀, d₅₀, d₉₀):

### Biological Test

The biological properties of compound (I) are investigated as follows, for example:

The inhibition of the EGF-R-mediated signal transmission can be demonstrated e.g. with cells which express human EGF-R and whose survival and proliferation depend on stimulation by EGF or TGF-alpha. A murine haematopoietic cell line is genetically modified so as to express functional human EGF-R. The proliferation of this cell line can therefore be stimulated by EGF.

The test is carried out as follows:

The cells are cultivated in RPMI/1640 medium. The proliferation is stimulated with 20 ng/ml of human EGF (Promega). To investigate the inhibitory activity of the compounds according to the invention these compounds are dissolved in 100% dimethylsulphoxide (DMSO) and added to the cultures in various dilutions, the maximum DMSO concentration being 1%. The cultures are incubated for 48 hours at 37° C.

In order to determine the inhibitory activity of the compound (I.1) according to the invention the relative cell number is measured in O.D. units using the Cell Titer 96TM AQueous Non-Radioactive Cell Proliferation Assay (Promega). The relative cell number is calculated as a percentage of the control and the concentration of active substance which inhibits the proliferation of the cells by 50% (IC50) is derived therefrom.

| | Inhibition of the EGFR- |
|---|---|
| | dependent proliferation |
| Compound | IC₅₀ [nM] |
| (I.1) | 10 |

### Indications

As was found, the compound of formula (I), particularly of formula (I.1), is characterised by its versatility in the therapeutic field. Particular mention should be made of the possible applications for which the compound of formula (I), particularly of formula (I.1), according to the invention may preferably be used on the basis of its pharmaceutical efficacy as a tyrosine inhibitor.

The compound of general formula (I) according to the invention thus inhibits signal transduction by tyrosine kinases, as demonstrated by the example of the human EGF receptor, and is therefore useful for treating pathophysiological processes caused by hyperfunction of tyrosine kinases. These are e.g. benign or malignant tumours, particularly tumours of epithelial and neuroepithelial origin, metastasisation and the abnormal proliferation of vascular endothelial cells (neoangiogenesis).

Compound (I) according to the invention is also useful for preventing and treating diseases of the airways and lungs which are accompanied by increased or altered production of mucus caused by stimulation of tyrosine kinases, e.g. in inflammatory diseases of the airways such as chronic bronchitis, chronic obstructive bronchitis, asthma, bronchiectasis, allergic or non-allergic rhinitis or sinusitis, cystic fibrosis, α1-antitrypsin deficiency, or coughs, pulmonary emphysema, pulmonary fibrosis and hyperreactive airways.

Compound (I) is also suitable for treating diseases of the gastrointestinal tract and bile duct and gall bladder which are associated with disrupted activity of the tyrosine kinases, such as may be found e.g. in chronic inflammatory changes such as cholecystitis, Crohn's disease, ulcerative colitis, and ulcers in the gastrointestinal tract or such as may occur in diseases of the gastrointestinal tract which are associated with increased secretions, such as Ménétrier's disease, secreting adenomas and protein loss syndrome.

In addition, compound (I) may be used to treat other diseases caused by abnormal function of tyrosine kinases, such as e.g. epidermal hyperproliferation (psoriasis), benign prostatic hyperplasia (BPH), inflammatory processes, diseases of the immune system, hyperproliferation of haematopoietic cells, the treatment of nasal polyps, etc.

### Combinations

The compounds of formula (I), particularly of formula (I.1), may be used on their own or in combination with other active substances. These combinations may be administered either simultaneously or sequentially. Optionally the compounds of formula (I.1) may also be used in combination with W, wherein W denotes a pharmacologically active substance and is selected (for example) from among the betamimetics, anticholinergics, corticosteroids, PDE4-inhibitors, LTD4-receptor (CysLT1, CysLT2, CysLT3) antagonists, LTB4-receptor (BLT1, BLT2) antagonists, inhibitors of MAP kinases such as for example p38, ERK1, ERK2, JNK1, JNK2, JNK3 or SAP, bradykinin (BK1, BK2) receptor antagonists, endothelin receptor antagonists, CXCR1 and/or CXCR2 receptor antagonists, and anti-tussive substances. In addition, double or triple combinations of W with the compounds of formula (I) may be combined. Examples of combinations of W with the compounds of formula 1 might be:
W denotes a betamimetic, combined with an anticholinergic, corticosteroid, PDE4-inhibitor, EGFR-inhibitor or LTD4-receptor antagonist,
W denotes an anticholinergic, combined with a betamimetic, corticosteroid, PDE4-inhibitor, EGFR-inhibitor or LTD4-receptor antagonist,
W denotes a corticosteroid, combined with a PDE4-inhibitor, EGFR-inhibitor or LTD4-receptor antagonist
W denotes a PDE4-inhibitor, combined with an EGFR-inhibitor or LTD4-receptor antagonist
W denotes an EGFR-inhibitor, combined with an anticholinergic.

Examples of betamimetics which may be used here preferably include compounds which are selected from among arformoterol, carmoterol, formoterol, indacaterol, salmeterol, albuterol, bambuterol, bitolterol, broxaterol, carbuterol, clenbuterol, fenoterol, hexoprenalin, ibuterol, isoetharin, isoprenalin, levosalbutamol, mabuterol, meluadrin, metaproterenol, milveterol, orciprenalin, pirbuterol, procaterol, reproterol, rimiterol, ritodrin, salmefamol, soterenol, sulphonterol, terbutalin, tiaramid, tolubuterol, zinterol and 6-hydroxy-8-{1-hydroxy-2-[2-(4-methoxy-phenyl)-1,1-dimethyl-ethylamino]-ethyl}-4H-benzo[1,4]oxazin-3-one, 8-{2-[2-(2,4-difluoro-phenyl)-1,1-dimethyl-ethylamino]-1-hydroxy-ethyl}-6-hydroxy-4H-benzo[1,4]oxazin-3-one, 8-{2-[2-(3,5-difluoro-phenyl)-1,1-dimethyl-ethylamino]-1-hydroxy-ethyl}-6-hydroxy-4H-benzo[1,4]oxazin-3-one, 8-{2-[2-(4-ethoxy-phenyl)-1,1-dimethyl-ethylamino]-1-hydroxy-ethyl}-6-hydroxy-4H-benzo[1,4]oxazin-3-one, 8-{2-[2-(4-fluoro-phenyl)-1,1-dimethyl-ethylamino]-1-hydroxy-ethyl}-6-hydroxy-4H-benzo[1,4]oxazin-3-one, N-(5-{2-[3-(4,4-diethyl-2-oxo-4H-benzo[d][1,3]oxazin-1-yl)-1,1-dimethyl-propylamino]-1-hydroxy-ethyl}-2-hydroxy-phenyl)-methanesulphonamide, N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methanesulphonamide, N-(5-{2-[3-(4,4-diethyl-6-methoxy-2-oxo-4H-benzo[d][1,3]oxazin-1-yl)-1,1-dimethyl-propylamino]-1-hydroxy-ethyl}-2-hydroxy-phenyl)-methanesulphonamide, N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methanesulphonamide, 8-{2-[1,1-dimethyl-3-(2-oxo-2,3-dihydro-benzimidazol-1-yl)-propylamino]-1-hydroxy-ethyl}-6-hydroxy-4H-benzo[1,4]oxazin-3-one, 8-{2-[1,1-dimethyl-3-(6-methyl-2-oxo-2,3-dihydro-benzimidazol-1-yl)-propylamino]-1-hydroxy-ethyl}-6-hydroxy-4H-benzo[1,4]oxazin-3-one, 8-{2-[1,1-dimethyl-3-(2-oxo-5-trifluoromethyl-2,3-dihydro-benzimidazol-1-yl)-propylamino]-1-hydroxy-ethyl}-6-hydroxy-4H-benzo[1,4]oxazin-3-one, 8-{2-[1,1-dimethyl-3-(3-methyl-2-oxo-2,3-dihydro-benzimidazol-1-yl)-propylamino]-1-hydroxy-ethyl}-6-hydroxy-4H-benzo[1,4]oxazin-3-one, N-[2-hydroxy-5-((1R)-1-hydroxy-2-{2-[4-(2-hydroxy-2-phenyl-ethylamino)-phenyl]-ethylamino}-ethyl)-phenyl]-formamide, 8-hydroxy-5-((1R)-1-hydroxy-2-{2-[4-(6-methoxy-biphenyl-3-ylamino)-phenyl]-ethylamino}-ethyl)-1H-quinolin-2-one, 8-hydroxy-5-[(1R)-1-hydroxy-2-(6-phenethylamino-hexylamino)-ethyl]-1H-quinolin-2-one, 5-[(1R)-2-(2-{4-[4-(2-amino-2-methyl-propoxy)-phenylamino]-phenyl}-ethylamino)-1-hydroxy-ethyl]-8-hydroxy-1H-quinolin-2-one, [3-(4-{6-[(2R)-2-hydroxy-2-(4-hydroxy-3-hydroxymethyl-phenyl)-ethylamino]-hexyloxy}-butyl)-5-methyl-phenyl]-urea, 4-((1R)-2-{6-[2-(2,6-dichloro-benzyloxy)-ethoxy]-hexylamino}-1-hydroxy-ethyl)-2-hydroxymethyl-phenol, 3-(4-{6-[(2R)-2-hydroxy-2-(4-hydroxy-3-hydroxymethyl-phenyl)-ethylamino]-hexyloxy}-butyl)-benzenesulphonamide, 3-(3-{7-[(2R)-2-hydroxy-2-(4-hydroxy-3-hydroxymethyl-phenyl)-ethylamino]-heptyloxy}-propyl)-benzenesulphonamide, 4-((1R)-2-{6-[4-(3-cyclopentanesulphonyl-phenyl)-butoxy]-hexylamino}-1-hydroxy-ethyl)-2-hydroxymethyl-phenol, N-1-adamantanyl-2-(3-[(2R)-2-({(2R)-2-hydroxy-2-[4-hydroxy-3-(hydroxymethyl)phenyl]ethyl}amino)propyl]phenyl}acetamide, (1R)-5-{2-[6-(2,2-difluoro-2-phenyl-ethoxy)-hexylamino]-1-hydroxy-ethyl}-8-hydroxy-1H-quinolin-2-one, (R,S)-4-(2-{[6-(2,2-difluoro-4-phenylbutoxy)hexyl]amino}-1-hydroxy-ethyl)-2-(hydroxymethyl)phenol, (R,S)-4-(2-{[6-(2,2-difluoro-2-phenylethoxy)hexyl]amino}-1-hydroxy-ethyl)-2-(hydroxymethyl)phenol, (R,S)-4-(2-{[4,4-difluoro-6-(4-phenylbutoxy)hexyl]amino}-1-hydroxy-ethyl)-2-(hydroxymethyl)phenol, (R,S)-4-(2-{[6-(4,4-difluoro-4-phenylbutoxy)hexyl]amino}-1-hydroxy-ethyl)-2-(hydroxymethyl)phenol, (R,S)-5-(2-{[6-(2,2-difluoro-2-phenylethoxy)hexyl]amino}-1-hydroxy-ethyl)-8-hydroxyquinolin-2(1H)-one, (R,S)-[2-({6-[2,2-difluoro-2-(3-methylphenyl)ethoxy]hexyl}amino)-1-hydroxyethyl]-2-(hydroxymethyl)phenol, 4-(1R)-2-{[6-(2,2-difluoro-2-phenylethoxy)hexyl]amino}-1-hydroxyethyl)-2-(hydroxymethyl)phenol, (R,S)-2-(hydroxymethyl)-4-(1-hydroxy-2-{[4,4,5I5-tetrafluoro-6-(3-phenylpropoxy)-hexyl]amino}ethyl)phenol, (R,S)-[5-(2-{[6-(2,2-difluoro-2-phenylethoxy)hexyl]amino}-1-hydroxy-ethyl)-2-hydroxyphenyl]formamide, (R,S)-4-[2-({6-[2-(3-bromophenyl)-2,2-difluoroethoxy]hexyl}amino)-1-hydroxyethyl]-2-(hydroxymethyl)phenol, (R,S)-N-[3-(1,1-difluoro-2-{[6-({2-hydroxy-2-[4-hydroxy-3-(hydroxymethyl)phenyl]-ethyl}amino)hexyl]oxy}ethyl)phenyl]-urea, 3-[3-(1,1-difluoro-2-{[6-({2-hydroxy-2-[4-hydroxy-3-(hydroxymethyl)phenyl]ethyl}amino)hexyl]oxy}ethyl)phenyl]imidazolidin-2,4-dione, (R,S)-4-[2-({6-[2,2-difluoro-2-(3-methoxyphenyl)ethoxy]hexyl}amino)-1-hydroxyethyl]-2-(hydroxymethyl)phenol, 5-((1R)-2-{[6-(2,2-difluoro-2-phenylethoxy)hexyl]amino}-1-hydroxyethyl)-8-hydroxyquinolin-2(1H)-one, 4-((1R)-2-{[4,4-difluoro-6-(4-phenylbutoxy)hexyl]amino}-1-hydroxy-ethyl)-2-(hydroxymethyl)phenol, (R,S)-4-(2-{[6-(3,3-difluoro-3-phenylpropoxy)hexyl]amino}-1-hydroxy-ethyl)-2-(hydroxymethyl)phenol, (R,S)-(2-{[6-(2,2-difluoro-2-phenylethoxy)-4,4-difluorohexyl]amino}-1-hydroxyethyl)-2-(hydroxymethyl)phenol, (R,S)-4-(2-{[6-(2,2-difluoro-3-phenylpropoxy)hexyl]amino}-1-hydroxy ethyl)-2-(hydroxymethyl)phenol, 3-[2-(3-chloro-phenyl)-ethoxy]-N-(2-diethylamino-ethyl)-N-{2-[2-(4-hydroxy-2-oxo-2,3-dihydro-benzothiazol-7-yl)-ethylamino]-ethyl}-propionamide, N-(2-diethylamino-ethyl)-N-{2-[2-(4-hydroxy-2-oxo-2,3-dihydro-benzothiazol-7-yl)-ethylamino]-ethyl}-3-(2-naphthalen-1-yl-ethoxy)-propionamide 7-[2-(2-{3-[2-(2-chloro-phenyl)-ethylamino]-propylsulphanyl}-ethylamino)-1-hydroxy-ethyl]-4-hydroxy-3H-benzothiazole-2-one,

optionally in the form of the racemates, enantiomers, diastereomers and optionally in the form of the pharmacologically acceptable acid addition salts, solvates or hydrates thereof. Preferably, according to the invention, the acid addition salts of the betamimetics are selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

Examples of anticholinergics which may be used here preferably include compounds which are selected from among: tiotropium salts, preferably the bromide salt, oxitropium salts, preferably the bromide salt, flutropium salts, preferably the bromide salt, ipratropium salts, preferably the bromide salt, aclidinium salts, preferably the bromide salt, glycopyrronium salts, preferably the bromide salt, trospium salts, preferably the chloride salt, tolterodine, (3R)-1-phenethyl-3-(9H-xanthen-9-carbonyloxy)-1-azoniabicyclo[2,2,2]octane-salts. In the above-mentioned salts the cations are the pharmacologically active constituents. As X⁻ anions the above-mentioned salts may preferably contain chloride, bromide, iodide, sulphate, phosphate, methanesulphonate, nitrate, maleate, acetate, citrate, fumarate, tartrate, oxalate, succinate, benzoate or p-toluenesulphonate, while chloride, bromide, iodide, sulphate, methanesulphonate or p-toluenesulphonate are preferred as counter-ions. Of all the salts the chlorides, bromides, iodides and methanesulphonates are particularly preferred.

Other specified compounds are: tropenol 2,2-diphenylpropionate methobromide, scopine 2,2-diphenylpropionate methobromide, scopine 2-fluoro-2,2-diphenylacetate methobromide, tropenol 2-fluoro-2,2-diphenylacetate methobromide, tropenol 3,3′,4,4′-tetrafluorobenzilate methobromide, scopine 3,3′,4,4′-tetrafluorobenzilate methobromide, tropenol 4,4′-difluorobenzilate methobromide, scopine 4,4′-difluorobenzilate methobromide, tropenol 3,3′-difluorobenzilate methobromide, scopine 3,3′-difluorobenzilate methobromide; tropenol 9-hydroxy-fluorene-9-carboxylate methobromide, tropenol 9-fluoro-fluorene-9-carboxylate methobromide, scopine 9-hydroxy-fluorene-9-carboxylate methobromide, scopine 9-fluoro-fluorene-9-carboxylate methobromide; tropenol 9-methyl-fluorene-9-carboxylate methobromide, scopine 9-methyl-fluorene-9-carboxylate methobromide, cyclopropyltropine benzilate methobromide, cyclopropyltropine 2,2-diphenylpropionate methobromide, cyclopropyltropine 9-hydroxy-xanthene-9-carboxylate methobromide, cyclopropyltropine 9-methyl-fluorene-9-carboxylate methobromide, cyclopropyltropine 9-methyl-xanthene-9-carboxylate methobromide, cyclopropyltropine 9-hydroxy-fluorene-9-carboxylate methobromide, cyclopropyltropine methyl 4,4′-difluorobenzilate methobromide, tropenol 9-hydroxy-xanthene-9-carboxylate methobromide, scopine 9-hydroxy-xanthene-9-carboxylate methobromide, tropenol 9-methyl-xanthene-9-carboxylate-methobromide, scopine 9-methyl-xanthene-9-carboxylate-methobromide, tropenol 9-ethyl-xanthene-9-carboxylate methobromide, tropenol 9-difluoromethyl-xanthene-9-carboxylate methobromide, scopine 9-hydroxymethyl-xanthene-9-carboxylate methobromide. The above-mentioned compounds may also be used as salts within the scope of the present invention, while instead of the methobromide, the metho-X salts may be used wherein X may have the meanings given hereinbefore for X⁻.

Compounds which may be used as corticosteroids are preferably those selected from among beclomethasone, betamethasone, budesonide, butixocort, ciclesonide, deflazacort, dexamethasone, etiprednol, flunisolide, fluticasone, loteprednol, mometasone, prednisolone, prednisone, rofleponide, triamcinolone, tipredane and pregna-1,4-diene-3.20-dione, 6-fluoro-11-hydroxy-16,17-[(1-methylethylidene)bis(oxy)]-21-[[4-[(nitrooxy)methyl]benzoyl]oxy]-(6-alpha,11-beta,16-alpha)-(9CI) (NCX-1024), 16,17-butylidenedioxy-6,9-difluoro-11-hydroxy-17-(methylthio)androst-4-en-3-one (RPR-106541), (S)-fluoromethyl 6,9-difluoro-17-[(2-furanylcarbonyl)oxy]-11-hydroxy-16-methyl-3-oxo-androsta-1,4-diene-17-carbothionate, (S)-(2-oxo-tetrahydro-furan-3S-yl) 6,9-difluoro-11-hydroxy-16-methyl-3-oxo-17-propionyloxy-androsta-1,4-dien-17-carbothionate, cyanomethyl 6-alpha,9-alpha-difluoro-11-beta-hydroxy-16alpha-methyl-3-oxo-17alpha-(2,2,3,3-tetramethylcyclopropylcarbonyl)oxy-androsta-1,4-diene-17beta-carboxylate,

optionally in the form of the racemates, enantiomers or diastereomers thereof and optionally in the form of the salts and derivatives thereof, the solvates and/or hydrates thereof. Any reference to steroids includes a reference to any salts or derivatives, hydrates or solvates thereof which may exist. Examples of possible salts and derivatives of the steroids may be: alkali metal salts, such as for example sodium or potassium salts, sulphobenzoates, phosphates, isonicotinates, acetates, dichloroacetates, propionates, dihydrogen phosphates, palmitates, pivalates or furoates.

PDE4-inhibitors which may be used are preferably compounds selected from among enprofyllin, theophyllin, roflumilast, ariflo (cilomilast), tofimilast, pumafentrin, lirimilast, apremilast, arofyllin, atizoram, oglemilastum, tetomilast, and 5-[(N-(2,5-dichloro-3-pyridinyl)-carboxamide]-8-methoxy-quinoline (D-4418), N-(3,5-dichloro-1-oxido-4-pyridinyl)-carboxamide]-8-methoxy-2-(trifluoromethyl)-quinoline (D-4396 (Sch-351591)), N-(3,5-dichloropyrid-4-yl)-[1-(4-fluorobenzyl)-5-hydroxy-indol-3-yl]glyoxylic acid amide (AWD-12-281 (GW-842470)), 9-[(2-fluorophenyl)methyl]-N-methyl-2-(trifluoromethyl)-9H-purin-6-amine (NCS-613), 4-[(2R)-2-[3-(cyclopentyloxy)-4-methoxyphenyl]-2-phenylethyl]-pyridine (CDP-840), N-[(3R)-3,4,6,7-tetrahydro-9-methyl-4-oxo-1-phenylpyrrolo[3,2,1-jk][1,4]benzodiazepin-3-yl]-4-pyridinecarboxamide (PD-168787), 4-[6,7-diethoxy-2,3-bis(hydroxymethyl)-1-naphthalenyl]-1-(2-methoxyethyl)-2(1H)-pyridinone (T-440), 2-[4-[6,7-diethoxy-2,3-bis(hydroxymethyl)-1-naphthalenyl]-2-pyridinyl]-4-(3-pyridinyl)-1(2H)-phthalazinone (T-2585), (3-(3-cyclopenyloxy-4-methoxybenzyl)-6-ethylamino-8-isopropyl-3H-purine (V-11294A), beta-[3-(cyclopentyloxy)-4-methoxyphenyl]-1,3-dihydro-1,3-dioxo-2H-isoindole-2-propanamide (CDC-801), imidazo[1,5-a]pyrido[3,2-e]pyrazin-6(5H)-one, 9-ethyl-2-methoxy-7-methyl-5-propyl-(D-22888), 5-[3-(cyclopentyloxy)-4-methoxyphenyl]-3-[(3-methylphenyl)methyl], (3S,5S)-2-piperidinone (HT-0712), 4-[1-[3,4-bis(difluoromethoxy)phenyl]-2-(3-methyl-1-oxido-4-pyridinyl)ethyl]-alpha,alpha-bis(trifluoromethyl)-benzenemethanol (L-826141), N-(3,5-dichloro-1-oxo-pyridin-4-yl)-4-difluoromethoxy-3-cyclopropylmethoxybenzamide, (−)p-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamide, (R)-(+)-1-(4-bromobenzyl)-4-[(3-cyclopentyloxy)-4-methoxyphenyl]-2-pyrrolidone, 3-(cyclopentyloxy-4-methoxyphenyl)-1-(4-N′-[N-2-cyano-5-methyl-isothioureido]benzyl)-2-pyrrolidone, cis[4-cyano-4-(3-cyclopentyloxy-4-methoxyphenyl)cyclohexane-1-carboxylic acid], 2-carbomethoxy-4-cyano-4-(3-cyclopropylmethoxy-4-difluoromethoxyphenyl)cyclohexan-1-one, cis[4-cyano-4-(3-cyclopropylmethoxy-4-difluoromethoxyphenyl)cyclohexan-1-ol], (R)-(+)-ethyl[4-(3-cyclopentyloxy-4-methoxyphenyl)pyrrolidin-2-ylidene]acetate, (S)-(−)-ethyl[4-(3-cyclopentyloxy-4-methoxyphenyl)pyrrolidin-2-ylidene]acetate, 9-cyclopentyl-5,6-dihydro-7-ethyl-3-(2-thienyl)-9H-pyrazolo[3,4-c]-1,2,4-triazolo[4,3-a]pyridine, 9-cyclopentyl-5,6-dihydro-7-ethyl-3-(tert-butyl)-9H-pyrazolo[3,4-c]-1,2,4-triazolo[4,3-a]pyridine,

optionally in the form of the racemates, enantiomers, diastereomers thereof and optionally in the form of the pharmacologically acceptable acid addition salts, solvates or hydrates thereof. According to the invention the preferred acid addition salts are selected from among hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

LTB4-receptor antagonists used here are preferably compounds selected from among for example ambulant (=ethyl[[4-[[3-[[4-[1-(4-hydroxyphenyl)-1-methylethyl]phenoxy]methyl]phenyl]methoxy]phenyl]iminomethyl]-carbamate), optionally in the form of the racemates, enantiomers, diastereomers thereof and optionally in the form of the pharmacologically acceptable acid addition salts, solvates, prodrugs or hydrates thereof. According to the invention the preferred acid addition salts are selected from among hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

LTD4-receptor antagonists used here are preferably compounds selected from among montelukast, pranlukast, zafirlukast, and (E)-8-[2-[4-[4-(4-fluorophenyl)butoxy]phenyl]ethenyl]-2-(1H-tetrazol-5-yl)-4H-1-benzopyran-4-one (MEN-91507), 4-[6-acetyl-3-[3-(4-acetyl-3-hydroxy-2-propylphenylthio)propoxy]-2-propylphenoxy]butyric acid (MN-001), 1-(((R)-(3-(2-(6,7-difluoro-2-quinolinyl)ethenyl)phenyl)-3-(2-(2-hydroxy-2-propyl)phenyl)thio)methylcyclopropaneacetic acid, 1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclopropaneacetic acid, [2-[[2-(4-tert-butyl-2-thiazolyl)-5-benzofuranyl]oxymethyl]phenyl]acetic acid optionally in the form of the racemates, enantiomers, diastereomers thereof and optionally in the form of the pharmacologically acceptable acid addition salts, solvates or hydrates thereof. According to the invention the preferred acid addition salts are selected from among hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

By salts or derivatives which the LTD4-receptor antagonists are optionally capable of forming are meant, for example: alkali metal salts, such as for example sodium or potassium salts, alkaline earth metal salts, sulphobenzoates, phosphates, isonicotinates, acetates, propionates, dihydrogen phosphates, palmitates, pivalates or furoates.

MAP Kinase inhibitors used are preferably compounds selected from among:

bentamapimod (AS-602801), doramapimod (BIRB-796), 5-carbamoylindole (SD-169), 6-[(aminocarbonyl)(2,6-difluorophenyl)amino]-2-(2,4-difluorophenyl)-3-pyridinecarboxamide (VX-702), alpha-[2-[[2-(3-pyridinyl)ethyl]amino]-4-pyrimidinyl]-2-benzothiazoleacetonitrile (AS-601245), 9,12-epoxy-1H-diindolo[1,2,3-fg:3′,2′,1′-kl]pyrrolo[3,4-i][1.6]benzodiazocine-10-carboxylic acid (CEP-1347), 4-[3-(4-chlorophenyl)-5-(1-methyl-4-piperidinyl)-1H-pyrazole-4-yl]-pyrimidine (SC-409),
optionally in the form of the racemates, enantiomers, diastereomers thereof and optionally in the form of the pharmacologically acceptable acid addition salts, prodrugs, solvates or hydrates thereof.

Bradykinin receptor antagonists that may be used are preferably compounds selected from among icatibant and 1-piperazinepentanaminium, delta-amino-4-[[4-[[[2,4-dichloro-3-[[(2,4-dim ethyl-8-quinolinyl)oxy]methyl]phenyl]sulphonyl]amino]tetrahydro-2H-pyran-4-yl]carbonyl]-N,N,N-trimethyl-ε-oxo, chloride, hydrochloride (1:1:1), (deltaS)-(MEN-16132), optionally in the form of the racemates, enantiomers and diastereomers thereof and optionally in the form of the pharmacologically acceptable acid addition salts, prodrugs, solvates or hydrates thereof.

Endothelin antagonists that may be used are preferably compounds selected from among actelion-1, ambrisentan, sitaxsentan, N-(2-acetyl-4.6-dimethylphenyl)-3-[[(4-chloro-3-methyl-5-isoxazolyl)amino]sulphonyl]-2-thiophenecarboxamide (TBC-3214) and bosentan, optionally in the form of the racemates, enantiomers and diastereomers thereof and optionally in the form of the pharmacologically acceptable acid addition salts, prodrugs, solvates or hydrates thereof.

Antitussive substances that may be used are preferably compounds selected from among hydrocodone, caramiphen, carbetapentane and dextramethorphan, optionally in the form of the racemates, enantiomers and diastereomers thereof and optionally in the form of the pharmacologically acceptable acid addition salts, prodrugs, solvates or hydrates thereof.

Substances of preferred CXCR1 and/or CXCR2 receptor antagonists that may be used are preferably compounds such as e.g. 3-[[3-[(dimethylamino)carbonyl]-2-hydroxyphenyl]amino]-4-[[(R)-1-(5-methylfuran-2-yl)propyl]amino]cyclobut-3-ene-1,2-dione (SCH-527123), optionally in the form of the racemates, enantiomers and diastereomers thereof and optionally in the form of the pharmacologically acceptable acid addition salts, prodrugs, solvates or hydrates thereof.

It is preferable, according to the invention, to use the acid addition salts of the above-mentioned betamimetics, anticholinergics, corticosteroids, PDE4 inhibitors, LTB4 (BLT1, BLT2) receptor antagonists, LTD4 (CysLT1, CysLT2, CysLT3) receptor antagonists, inhibitors of MAP kinases such as for example p38, ERK1, ERK2, JNK1, JNK2, JNK3 or SAP, bradykinin receptor antagonists, endothelin receptor antagonists, antitussive substances, CXCR1 and/or CXCR2 receptor antagonists also selected from among hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

### Other Pharmaceutical Compositions

The compound (I) according to the invention may be administered by oral, transdermal, inhalative, parenteral or sublingual route, preferably inhalative. It is present as an active ingredient in conventional preparations, for example in compositions consisting essentially of an inert pharmaceutical carrier and an effective dose of the active substance, such as for example tablets, coated tablets, capsules, lozenges, powders, solutions, suspensions, emulsions, syrups, suppositories, transdermal systems etc. For inhalation, solutions or powders, preferably powders, containing 1 to 100% of active substance are suitable according to the invention. Possible powder formulations may be (1) mixtures of jet-ground active substance and a carrier adjuvant or (2) micronisates consisting of spray-dried particles from solutions with active substance and/or adjuvant or (3) pure jet-ground particles of active substance. In the case of powder mixtures the formulations preferably contain 10 to 50%, particularly preferably 20 to 40% of active substance. In the case of formulations obtained from spray-dried particles, the formulations contain 25 to 100%, preferably 40-60 of active substance. A particle size distribution that is suitable for inhalation is between 1 and 5 μm aerodynamic particle size distribution. (P. R. Byron, Drug Dev. Ind. Pharm. 12:993-1015 (1986)/G. Scheuch, Adv. Drug Del. Systems 996-1008 (2006))

The following Examples illustrate the present invention without, however, restricting its scope:

### Pharmaceutical Formulation Example 1

### Capsules for Powder Inhalation Containing 5 mg of Active Substance, for Example Compound (I.1),

1 capsule contains:

| | |
|---|---|
| Active substance (jet-ground) | 5.0 mg |
| Lactose for inhalation purposes | 15.0 mg |
| | 20.0 mg |

### Preparation:

The active substance is mixed with lactose for inhalation purposes. The mixture is packed into capsules in a capsule machine (weight of empty capsule approx. 100 mg).

Capsule weight: 120.0 mg
Capsule size: 3

### Pharmaceutical Formulation Example 2

### Capsules for Powder Inhalation Containing 7.5 mg of Active Substance, for Example Compound (I.1),

1 capsule contains:

| | |
|---|---|
| Active substance (jet-ground) | 7.5 mg |
| Lactose (ground) | 7.5 mg |
| Lactose for inhalation purposes | 15.0 mg |
| | 30.0 mg |

### Preparation:

The active substance is mixed with lactose (ground) and then with lactose for inhalation purposes. The mixture is packed into capsules in a capsule making machine (weight of empty capsule about 100 mg).

Weight of capsule: 130.0 mg
Capsule size: 3

### Pharmaceutical Formulation Example 3

### Capsules for Powder Inhalation Containing 10 mg of Active Substance, for Example Compound (I.1),

1 capsule contains:

| | |
|---|---|
| Active substance (jet-ground) | 10.0 mg |
| | 10.0 mg |

### Preparation:

The active substance, for example compound (I.1), is jet-ground and packed into capsules in a capsule machine (weight of empty capsule approx. 100 mg).

Capsule weight: 110.0 mg
Capsule size: 3

### Pharmaceutical Formulation Example 4

### Capsules for Powder Inhalation Containing 10 mg of Active Substance, for Example Compound (I.1),

1 capsule contains:

| | |
|---|---|
| Active substance (spray-dried) | 10.0 mg |
| | 10.0 mg |

### Preparation:

The active substance, for example compound (I.1), is jet-ground and packed into capsules in a capsule machine (weight of empty capsule approx. 100 mg).

Capsule weight: 110.0 mg
Capsule size: 3

### Pharmaceutical Formulation Example 5

### Capsules for Powder Inhalation Containing 5 mg of Active Substance, for Example Compound (I.1),

1 capsule contains:

| | |
|---|---|
| active substance (5 mg) and adjuvant (5 mg) | 10.0 mg |
| (spray-dried together) | |

### Preparation:

The active substance and the adjuvant are dissolved in a solvent and spray-dried. The resulting powder is packaged using a capsule machine (weight of empty capsule approx. 100 mg).

Capsule weight: 110.0 mg
Capsule size: 3
Example of a Formulation with Preferred Properties and Testing Thereof

A powder mixture of 25% jet-ground active substance particles, for example compound (I.1), and lactose (200M) as carrier adjuvant is prepared according to Formulation Example 1, packed into capsules and tested using the HandiHaler:

The inhalable fraction which constitutes the fraction of particles obtained from the HandiHaler that have an aerodynamic particle size distribution of less than 5 μm (Guidance for Industry “Metered Dose Inhaler (MDI) and Dry Powder Inhaler (DPI) Drug Products; CDER, FDA, U.S. Dept of Health and Human Services; October 1998), is determined using the Next Generation Impactor (Apparatus 5; USP-NF General Chapter <601>). The chemical identity of the active substance or its content on the respective precipitation stage of the impactor is determined by liquid chromatography:

Inhalable fraction (4 kPa): 1.84 mg
Inhalable fraction (1 kPa): 1.36 mg
Flow rate dependency: 0.74

### APPENDIX

TABLE 1
Powder X-ray peaks (up to 30° 2 Θ) including normalised
intensities of the anhydrous form of compound (6)

| 2 Θ | dₕₖₗ | |
|---|---|---|
| [°] | [Å] | I/Iₒ |
| 6.97 | 12.68 | 5 |
| 7.81 | 11.32 | 23 |
| 9.14 | 9.67 | 13 |
| 10.22 | 8.65 | 27 |
| 11.96 | 7.39 | 11 |
| 12.35 | 7.16 | 29 |
| 13.00 | 6.81 | 4 |
| 13.37 | 6.62 | 5 |
| 13.94 | 6.35 | 4 |
| 14.20 | 6.23 | 11 |
| 14.96 | 5.92 | 10 |
| 15.63 | 5.66 | 89 |
| 16.62 | 5.33 | 40 |
| 17.38 | 5.10 | 32 |
| 17.84 | 4.97 | 11 |
| 18.00 | 4.92 | 32 |
| 18.12 | 4.89 | 51 |
| 18.31 | 4.84 | 10 |
| 18.63 | 4.76 | 86 |
| 19.22 | 4.61 | 20 |
| 19.39 | 4.57 | 35 |
| 19.92 | 4.45 | 7 |
| 20.54 | 4.32 | 3 |
| 20.81 | 4.27 | 11 |
| 21.35 | 4.16 | 16 |
| 21.85 | 4.07 | 28 |
| 22.43 | 3.96 | 14 |
| 22.66 | 3.92 | 100 |
| 22.92 | 3.88 | 18 |
| 23.33 | 3.81 | 7 |
| 23.54 | 3.78 | 6 |
| 23.82 | 3.73 | 6 |
| 24.05 | 3.70 | 12 |
| 24.33 | 3.65 | 9 |
| 24.89 | 3.57 | 4 |
| 25.76 | 3.46 | 3 |
| 26.14 | 3.41 | 4 |
| 26.70 | 3.34 | 9 |
| 26.89 | 3.31 | 37 |
| 27.56 | 3.23 | 2 |
| 28.44 | 3.14 | 4 |
| 29.39 | 3.04 | 5 |
| 30.15 | 2.96 | 16 |

TABLE 2
Indexed powder X-ray peaks (up to 30° 2 Θ) including normalised
intensities of compound (I.2)

| 2 Θ | dₕₖₗ | | | indexation | | 2 Θ_{obs} − 2 Θ_{calc} |
|---|---|---|---|---|---|---|
| [°] | [Å] | I/Iₒ | h | k | l | [°] |
| 7.44 | 11.87 | 18 | 0 | 2 | 0 | −0.002 |
| 8.39 | 10.52 | 6 | 1 | 0 | 0 | −0.002 |
| 9.20 | 9.60 | 44 | 1 | 1 | 0 | 0.017 |
| 10.15 | 8.71 | 3 | −1 | 1 | 1 | 0.033 |
| 11.25 | 7.86 | 12 | 1 | 2 | 0 | 0.02 |
| 11.44 | 7.73 | 10 | 0 | 2 | 1 | −0.003 |
| 12.02 | 7.36 | 17 | −1 | 2 | 1 | 0.011 |
| 14.01 | 6.32 | 46 | 1 | 3 | 0 | 0.014 |
| 14.90 | 5.94 | 27 | 0 | 4 | 0 | −0.018 |
| 16.08 | 5.51 | 25 | −2 | 1 | 1 | 0.026 |
| 16.49 | 5.37 | 2 | −1 | 1 | 2 | −0.004 |
| 16.85 | 5.26 | 3 | 2 | 0 | 0 | 0.007 |
| 17.39 | 5.10 | 31 | 0 | 0 | 2 | −0.008 |
| 17.72 | 5.00 | 53 | −1 | 2 | 2 | <0.001 |
| 18.16 | 4.88 | 39 | 1 | 3 | 1 | 0.002 |
| 18.42 | 4.81 | 13 | 2 | 2 | 0 | −0.009 |
| 18.95 | 4.68 | 100 | 0 | 2 | 2 | 0.002 |
| 19.23 | 4.61 | 28 | −2 | 3 | 1 | −0.008 |
| 19.62 | 4.52 | 36 | −1 | 3 | 2 | 0.008 |
| 20.30 | 4.37 | 11 | −2 | 2 | 2 | −0.014 |
| 20.51 | 4.33 | 13 | 1 | 5 | 0 | 0.002 |
| 20.63 | 4.30 | 24 | 0 | 5 | 1 | <0.0010 |
| 20.95 | 4.24 | 13 | −1 | 5 | 1 | −0.001 |
| 21.65 | 4.10 | 2 | −2 | 4 | 1 | −0.011 |
| 22.00 | 4.04 | 10 | −2 | 3 | 2 | 0.017 |
| 22.16 | 4.01 | 12 | 2 | 1 | 1 | 0.006 |
| 22.99 | 3.87 | 17 | 0 | 4 | 2 | −0.001 |
| 23.63 | 3.76 | 4 | −3 | 1 | 1 | 0.001 |
| 24.50 | 3.63 | 48 | −3 | 0 | 2 | 0.015 |
| 24.78 | 3.59 | 11 | −3 | 1 | 2 | 0.005 |
| 25.36 | 3.51 | 8 | 3 | 0 | 0 | −0.014 |
| 25.63 | 3.47 | 62 | −3 | 2 | 2 | <0.001 |
| 26.50 | 3.36 | 3 | 3 | 2 | 0 | 0.024 |
| 26.83 | 3.32 | 14 | 1 | 4 | 2 | 0.006 |
| 26.99 | 3.30 | 25 | −3 | 3 | 2 | 0.001 |
| 27.43 | 3.25 | 9 | −2 | 3 | 3 | −0.006 |
| 27.72 | 3.22 | 11 | 0 | 7 | 1 | 0.019 |
| 28.51 | 3.13 | 3 | −1 | 4 | 3 | 0.011 |
| 28.73 | 3.11 | 8 | −3 | 1 | 3 | −0.005 |
| 29.50 | 3.03 | 4 | −3 | 2 | 3 | 0.03 |
| 30.02 | 2.97 | 8 | 1 | 7 | 1 | 0.008 |

TABLE 3
Powder X-ray peaks (up to 30° 2 Θ) including
normalised intensities of compound (I.1)

| 2 Θ | dₕₖₗ | |
|---|---|---|
| [°] | [Å] | I/Iₒ |
| 5.32 | 16.58 | 72 |
| 7.10 | 12.43 | 38 |
| 8.04 | 10.99 | 12 |
| 9.59 | 9.22 | 28 |
| 10.29 | 8.59 | 28 |
| 10.65 | 8.30 | 11 |
| 11.62 | 7.61 | 30 |
| 13.89 | 6.37 | 32 |
| 14.68 | 6.03 | 21 |
| 15.39 | 5.75 | 46 |
| 15.62 | 5.67 | 14 |
| 16.12 | 5.50 | 69 |
| 16.70 | 5.30 | 92 |
| 17.36 | 5.10 | 28 |
| 18.54 | 4.78 | 27 |
| 19.01 | 4.66 | 100 |
| 19.20 | 4.62 | 81 |
| 19.36 | 4.58 | 24 |
| 19.77 | 4.49 | 34 |
| 20.57 | 4.31 | 32 |
| 20.92 | 4.24 | 72 |
| 21.61 | 4.11 | 55 |
| 21.84 | 4.07 | 26 |
| 22.06 | 4.03 | 53 |
| 22.36 | 3.97 | 27 |
| 22.59 | 3.93 | 18 |
| 23.02 | 3.86 | 10 |
| 23.25 | 3.82 | 9 |
| 23.39 | 3.80 | 11 |
| 23.85 | 3.73 | 12 |
| 24.05 | 3.70 | 27 |
| 24.50 | 3.63 | 15 |
| 24.74 | 3.60 | 6 |
| 25.05 | 3.55 | 17 |
| 25.25 | 3.52 | 8 |
| 25.81 | 3.45 | 60 |
| 26.37 | 3.38 | 23 |
| 26.80 | 3.32 | 65 |
| 27.07 | 3.29 | 72 |
| 27.61 | 3.23 | 36 |
| 27.97 | 3.19 | 7 |
| 28.16 | 3.17 | 17 |
| 28.40 | 3.14 | 14 |
| 28.68 | 3.11 | 8 |
| 28.92 | 3.08 | 26 |
| 29.05 | 3.07 | 23 |
| 29.42 | 3.03 | 8 |
| 29.96 | 2.98 | 7 |

TABLE 4
Powder X-ray peaks (up to 30° 2 Θ) including normalised
intensities of the low-melting form of compound (I.3)

| 2 Θ | dₕₖₗ | |
|---|---|---|
| [°] | [Å] | I/Iₒ |
| 4.20 | 21.02 | 100 |
| 9.43 | 9.37 | 37 |
| 10.09 | 8.76 | 6 |
| 12.46 | 7.10 | 27 |
| 13.52 | 6.54 | 15 |
| 14.29 | 6.19 | 3 |
| 15.35 | 5.77 | 4 |
| 15.92 | 5.56 | 13 |
| 16.38 | 5.41 | 9 |
| 16.83 | 5.26 | 9 |
| 17.93 | 4.94 | 32 |
| 18.54 | 4.78 | 32 |
| 19.49 | 4.55 | 6 |
| 20.29 | 4.37 | 6 |
| 21.25 | 4.18 | 8 |
| 21.65 | 4.10 | 11 |
| 22.32 | 3.98 | 2 |
| 22.59 | 3.93 | 3 |
| 22.89 | 3.88 | 6 |
| 24.80 | 3.59 | 11 |
| 25.85 | 3.44 | 41 |
| 26.18 | 3.40 | 32 |
| 27.05 | 3.29 | 4 |
| 27.58 | 3.23 | 9 |
| 27.81 | 3.21 | 16 |

TABLE 5
Powder X-ray peaks (up to 30° 2 Θ) including normalised
intensities of the high-melting form of compound (I.3)

| 2 Θ | dₕₖₗ | |
|---|---|---|
| [°] | [Å] | I/Iₒ |
| 4.68 | 18.89 | 100 |
| 9.33 | 9.47 | 3 |
| 9.69 | 9.12 | 36 |
| 11.01 | 8.03 | 10 |
| 11.72 | 7.55 | 11 |
| 13.94 | 6.35 | 2 |
| 14.80 | 5.98 | 17 |
| 15.09 | 5.86 | 48 |
| 15.50 | 5.71 | 12 |
| 16.18 | 5.47 | 17 |
| 17.91 | 4.95 | 12 |
| 18.30 | 4.84 | 3 |
| 18.51 | 4.79 | 7 |
| 18.92 | 4.69 | 5 |
| 19.40 | 4.57 | 15 |
| 21.11 | 4.20 | 9 |
| 21.48 | 4.13 | 5 |
| 22.14 | 4.01 | 12 |
| 22.94 | 3.87 | 9 |
| 23.09 | 3.85 | 8 |
| 23.34 | 3.81 | 37 |
| 23.98 | 3.71 | 24 |
| 24.26 | 3.67 | 11 |
| 24.69 | 3.60 | 6 |
| 25.31 | 3.52 | 37 |
| 25.81 | 3.45 | 2 |
| 26.74 | 3.33 | 4 |
| 27.96 | 3.19 | 21 |
| 28.69 | 3.11 | 4 |
| 29.27 | 3.05 | 3 |
| 29.62 | 3.01 | 5 |

TABLE 6
Solubility of the different crystalline
forms of compound (I) and (6) in water

| | solubility | intrinsic pH of the |
|---|---|---|
| form | [mg/ml] | saturated solution |
| anhydrous free base (6) | <0.001 | 5.9 |
| monohydrate free base (I.2) | <0.001 | 5.9 |
| hemihydrochloride | 0.17 | 6.5 |
| sesquihydrate (I.1) | | |
| low-melting form | 0.047 | 4.0 |
| monohydrochloride (I.3) | | |
| high-melting form | 0.041 | 3.3 |
| monohydrochloride (I.3) | | |

1. A compound of formula (I) 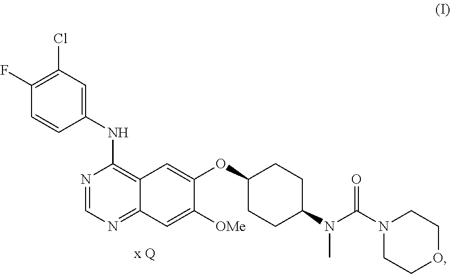
wherein x Q denotes x H₂O, x HCl; or x 0.5 HCl x 1.5 H₂O.

2. The compound according to claim 1 of formula (I.1)

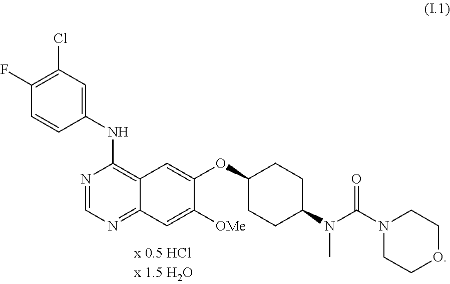

3. The compound according to claim 2 wherein the compound of formula (I.1) is in a crystalline form, having a reflection in the X-ray powder diagram occur at the dₕₖₗ value of 4.66 Å.

4. The compound according to claim 2 wherein the compound of formula (I.1) is in a crystalline form, having reflections in the X-ray powder diagram occur at dₕₖₗ values of 16.58 Å, 5.50 Å, 5.30 Å, 4.66 Å, 4.62 Å, 4.24 Å and 3.45 Å; or
an X-ray diffraction pattern comprising peaks at 5.32, 16.12, 16.70, 19.01, 19.20, 20.92 and 25.81 degrees 2θ (±0.5 degrees 2θ) when measured using CuKα radiation.

5. A method for the treatment of inflammatory or allergic diseases of the airways comprising administering to a patient in need thereof a therapeutically effective amount of a compound according to claim 1.

6. A method for the treatment of COPD and/or chronic bronchitis comprising administering to a patient in need thereof a therapeutically effective amount of a compound according to claim 1.

7. A process for the stereoselective preparation of a compound of formula (I.1) comprising recrystallization of the compound (I.2) from a suitable solvent and aqueous hydrochloric acid to obtain the compound of formula (I.1)

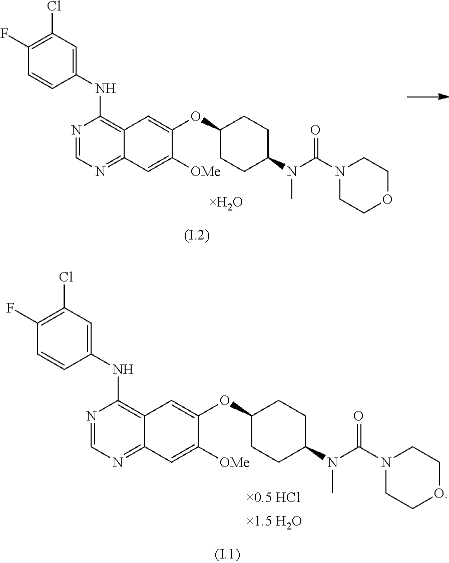

8. The process according to claim 7 further comprising steps (A) to (D) for the stereoselective preparation of the compound of formula (I.2), wherein
(A) denotes the hydrogenation of a compound of formula (I) 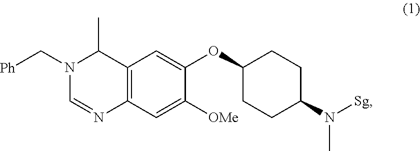
to obtain a compound of formula (2), 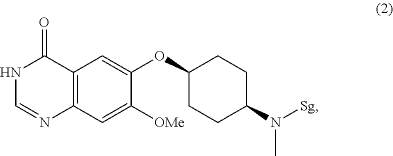
(B) denotes the reaction of a compound of formula (2) 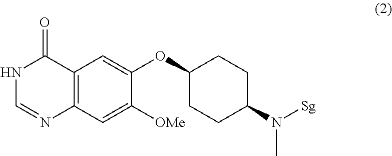
with a chlorinating agent to provide the intermediate of formula (3) 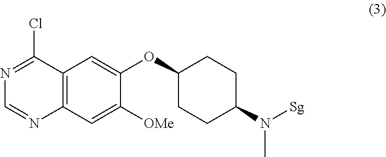
followed by reaction with 3-chloro-4-fluoroaniline to obtain the compound of formula (4) 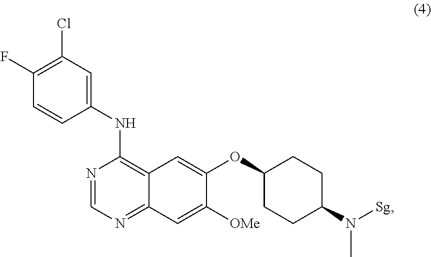
(C) denotes the deprotection of the compound of formula (4), optionally after a preceding purification step, to obtain a compound of formula (5.1) or (5.2), 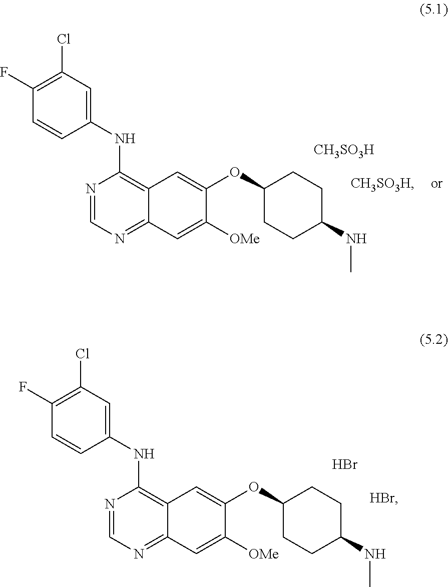
wherein deprotection with methanesulfonic acid provides (5.1) and deprotection with hydrogen bromide provides (5.2), and
(D) denotes the reaction of the compound of formula (5.1) or (5.2) with morpholine-4-carbonyl chloride, to obtain a compound of formula (I.2) 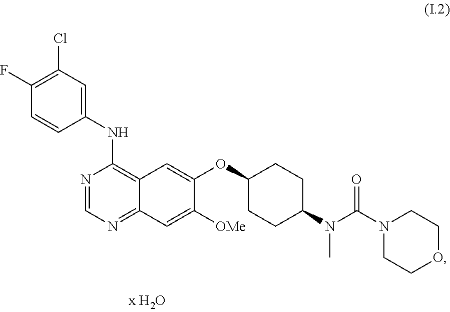
wherein steps (A) to (D) take place successively in the order specified.

9. The process according to claim 7 further comprising an additional recrystallisation of the compound of formula (I.1) from a suitable solvent.

10. The process according to claim 7 further comprising steps (A), (B), (C), (F) and (G) for the stereoselective preparation of the compound of formula (I.2), wherein
(A) denotes the hydrogenation of a compound of formula (I) 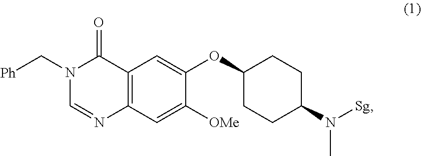
to obtain a compound of formula (2), 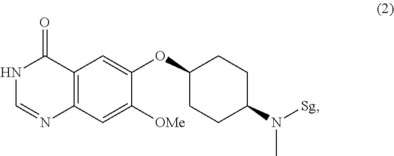
(B) denotes the reaction of a compound of formula (2) 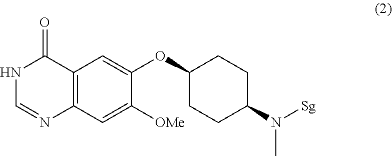
with a chlorinating agent to provide the intermediate of formula (3) 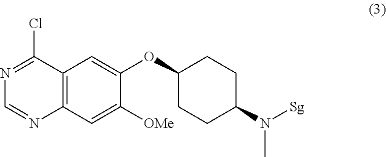
followed by reaction with 3-chloro-4-fluoroaniline to obtain the compound of formula (4) 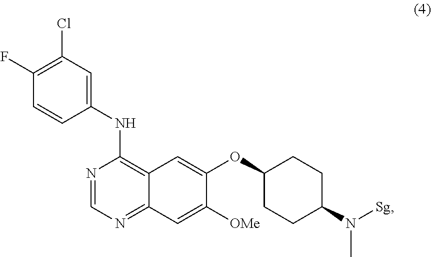
(C) denotes the deprotection of the compound of formula (4), optionally after a preceding purification step, to obtain a compound of formula (5.1) or (5.2) 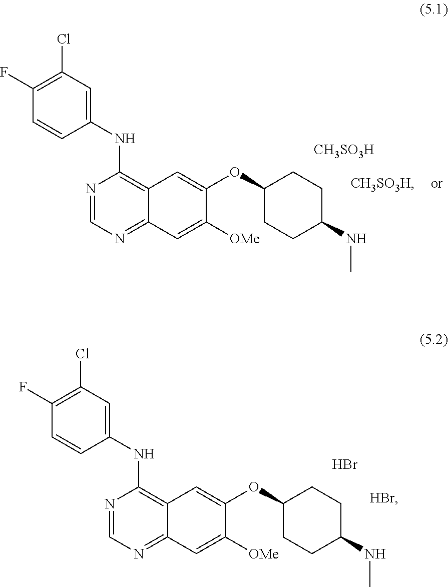
wherein deprotection with methanesulfonic acid provides (5.1) and deprotection with hydrogen bromide provides (5.2),
(F) denotes the reaction of the compound of formula (5.1) or (5.2) with bis-[1,2,4]triazol-1-yl-methanone to form a compound of formula (7), 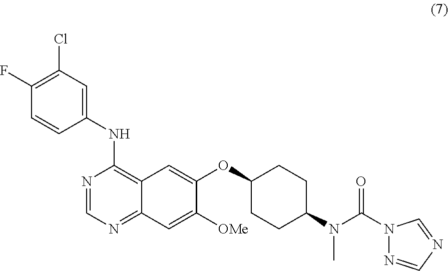
and
(G) denotes the reaction of the compound of formula (7) with morpholine to form the compound of formula (I.2) 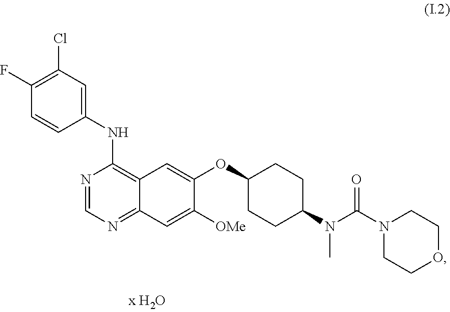
wherein steps (A), (B), (C), (F) and (G) take place successively in the order specified.

11. A compound of formula (5.1) or (5.2)

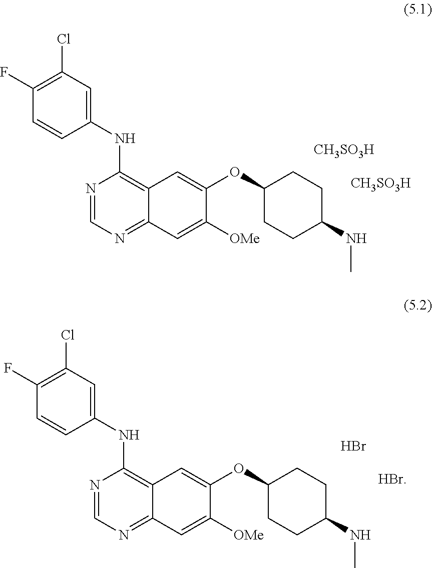

12. The crystalline form of the compound (6) 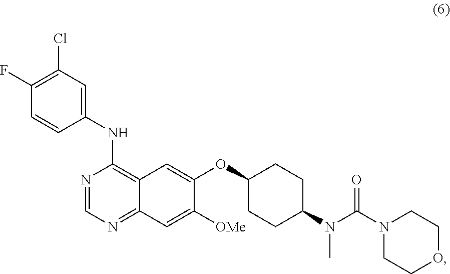
characterised in that a reflection in the X-ray powder diagram occurs at the dₕₖₗ value of 3.92 Å.

13. The crystalline form of the compound (6) 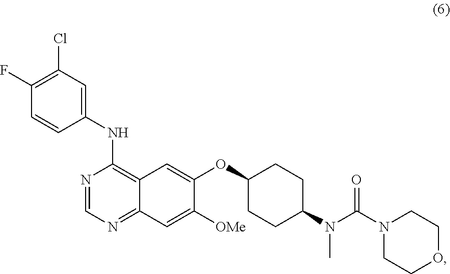
characterised in that reflections in the X-ray powder diagram occur at dₕₖₗ values of 5.66 Å, 5.33 Å, 4.89 Å, 4.76 Å, 3.92 Å and 3.31 Å; or
an X-ray diffraction pattern comprising peaks at 15.63, 16.62, 18.12, 18.63, 22.66 and 26.89 degrees 2θ (±0.5 degrees 2θ) when measured using CuKα radiation.

14. A pharmaceutical composition comprising a compound of formula (I) according to claim 1.

15. The pharmaceutical composition according to claim 15 further comprising lactose.

16. A pharmaceutical combination comprising one or more compounds of formula (I) according to claim 1 and one or more compounds that are selected from among the categories of the betamimetics, anticholinergics, corticosteroids, PDE4 inhibitors, LTD4 receptor antagonists, LTB4 receptor antagonists, inhibitors of MAP kinases, bradykinin receptor antagonists, endothelin receptor antagonists, CXCR1 and/or CXCR2 receptor antagonists, and anti-tussive substances, or double or triple combinations thereof.
